(19) United States
(12) Patent Application Publication
(10) Pub. No.: US 2016/0303089 A1
(43) Pub. Date: Oct. 20, 2016

(54) COMPOUND PERTAINING TO NEUROPOIESIS AND DRUG COMPOSITION

(71) Applicant: KYOTO UNIVERSITY, Kyoto-shi, Kyoto (JP)

(72) Inventors: Masatoshi HAGIWARA, Kyoto-shi, Kyoto (JP); Akiko KOBAYASHI, Kyoto-shi, Kyoto (JP)

(73) Assignee: KYOTO UNIVERSITY, Kyoto-shi, Kyoto (JP)

(21) Appl. No.: 15/101,862

(22) PCT Filed: Dec. 3, 2014
(86) PCT No.: PCT/JP2014/082035

§ 371 Date: Jun. 3, 2016

(30) Foreign Application Priority Data
Dec. 5, 2013 (JP) 2013-252443

(51) Int. Cl. A61K 31/429; C12N 5/0793; C12N 5/0797; A61K 31/695

(52) U.S. Cl. CPC A61K 31/429; A61K 31/695; C12N 5/0619; C12N 5/0623

(57) ABSTRACT

A composition for activating neurogenesis or the growth of neurons is provided.

In one or more embodiments, a composition contains as an active ingredient a compound with a DYRK inhibitory capacity or a prodrug thereof or a pharmaceutically acceptable salt thereof. In one or more embodiments, a composition contains as an active ingredient a compound expressed by the following general formula (I) and/or (II) or prodrug thereof or a pharmaceutically acceptable salt thereof.

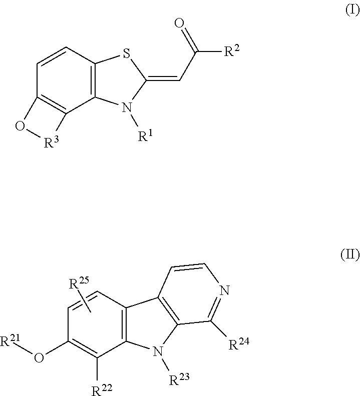

### TECHNICAL FIELD

The present disclosure relates to a compound and a pharmaceutical composition for neurogenesis. The present disclosure also relates to the activation of neurogenesis and/or the activation of growth of neurons.

### BACKGROUND ART

In recent years, it has become clear that nerves may be newly formed or regenerated in the central nervous system. This has encouraged the development of drugs that can control neurogenesis. Patent Document 1 discloses a neurogenesis promoter that contains a peptide capable of promoting neurogenesis in the hippocampus of the mammalian brain. Patent Document 2 discloses a low molecular weight compound that has neurogenesis activity.

### PRIOR ART DOCUMENTS

### Patent Documents

Patent Document 1: JP 2010-105996 A

Patent Document 2: JP 2009-292782 A

### DISCLOSURE OF INVENTION

### Problem to be Solved by the Invention

In one aspect, the present disclosure provides a composition for activating neurogenesis, for growing neurons, or for inhibiting differentiation of neurons.

### Means for Solving Problem

In one or more embodiments, the present disclosure relates to a composition for activating neurogenesis or the growth of neurons. The composition contains as an active ingredient a compound with a DYRK inhibitory capacity or a prodrug thereof or a pharmaceutically acceptable salt thereof.

In one or more embodiments, the present disclosure relates to a composition for activating neurogenesis or the growth of neurons. The composition contains as an active ingredient a compound expressed by the following general formula (I) and/or (II) or a prodrug thereof or a pharmaceutically acceptable salt thereof.

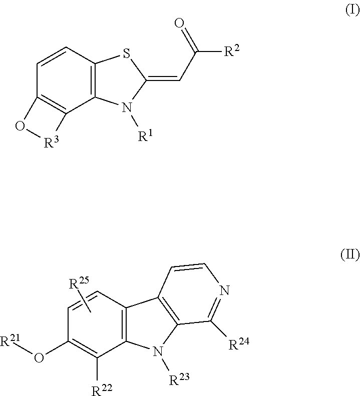

(where, in the general formula (I), R¹ and R² each independently represent a hydrogen atom or a C₁₋₆ hydrocarbon chain,

R³ represents

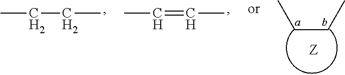

where Z and atoms marked with a and b form a ring selected from the group consisting of one benzene ring, one heteroaromatic ring, an aromatic ring in which one or more benzene rings are condensed, a heteroaromatic ring in which one or more heteroaromatic rings are condensed, a mixed condensed polycyclic ring in which one or more benzene rings are condensed with one or more heteroaromatic rings, and a cyclic aliphatic, and the ring may have at least one substituent that is a hydrogen atom, a halogen atom, or a C₁₋₆ alkyl group, and

R⁴ represents a hydrogen atom, a halogen atom, or a C₁₋₆ alkyl group, and

where, in the general formula (ID, R²¹ and R²³ each independently represent a hydrogen atom, a linear, branched, or cyclic C₁₋₆ alkyl group, a benzyl or heteroarylmethyl group, a substituted or unsubstituted aryl group, or a substituted or unsubstituted heteroaryl group,

R²² is selected from the group consisting of —R²⁶, —C≡C—R²⁶, —CH═CH—R²⁶, and —O—(CH₂)n-R²⁶, where n is 1 to 6, R²⁶ is selected from the group consisting of a hydrogen atom, a hydroxyl group, a C₁₋₈ alkyl group, —Si(R²⁷)₃, a substituted or unsubstituted phenyl group, a monocyclic heteroaromatic ring group, and a cyclic aliphatic group, R²⁷ represents a hydrogen atom, a C₁₋₆ alkyl group, a trihalomethyl group, or a hydroxyl group, and three elements represented by R²⁷ of —Si(R²⁷)₃ may differ from each other, alternatively R²² is bonded with R²¹ to form a ring, and —R²¹-R²²— is selected from the group consisting of —(CH₂)m-CH₂—, —CH═CH—, —(CH₂)m-O—, halogen-substituted —(CH₂)m-CH₂—, halogen-substituted —CH═CH—, and halogen-substituted —(CH₂)m-O—, where m is 1 to 6, and

R²⁴ and R²⁵ represent a hydrogen atom or a C₁₋₆ alkyl group.)

In one or more embodiments, the present disclosure relates to a method for activating neurogenesis, which includes administering the composition of the present disclosure to a subject. In one or more embodiments, the present disclosure relates to a method for preparing neurons, which includes culturing neurons in a culture medium containing the composition of the present disclosure.

### BRIEF DESCRIPTION OF DRAWINGS

FIG. 1 illustrates that the continuous oral administration of a compound 2 to animal individuals (mice) activates neurogenesis in the dentate gyrus of the hippocampus.

FIG. 2 illustrates an experimental system that demonstrates the activation of growth of neural stem cells.

FIG. 3 is an example of a graph showing the results of analyzing the ratio of BrdU positive cells in the cultured neural stem cells by ArrayScan after the administration of compounds 1 to 3.

FIG. 4 is an example of the results of detecting the expression of cyclin D1 in the cultured neural stem cells by western blotting after the administration of compounds 1 to 3.

FIG. 5 illustrates an experimental system that demonstrates the effect of the activation of neurogenesis by suppressing the expression of DYRK1A.

FIG. 6 is an example of a graph showing the results of analyzing the ratio of BrdU positive cells in the cultured neural stem cells by ArrayScan after the administration of shRNA that suppresses the expression of DYRK1A.

FIG. 7 is an example of the results of detecting the expression of cyclin D1 in the cultured neural stem cells by western blotting after the administration of shRNA that suppresses the expression of DYRK1A.

FIG. 8 is an example of the results of detecting the expression of cyclin D1 in neurons by western blotting after inducing the expression of DYRK family and adding a compound 2.

### DESCRIPTION OF THE INVENTION

[Compound with DYRK Inhibitory Capacity/CLK Inhibitory Capacity]

“DYRK” in the present disclosure means a kinase that belongs to the dual-specificity tyrosine phosphorylation-regulated kinase family. “CLK” in the present disclosure means a kinase that belongs to the CDC-like kinase family. In one or more embodiments, an “inhibitory capacity” in the present disclosure means the capacity to inhibit the activity of a kinase.

In one of more embodiments, a compound with a DYRK inhibitory capacity means that the compound has an inhibitory capacity for at least one kinase that belongs to the DYRK family. In another one or more embodiments, a compound with a DYRK inhibitory capacity means that the compound has activity to inhibit at least one phosphorylation activity of a kinase that belongs to the DYRK family. In one or more embodiments, the compound with a DYRK inhibitory capacity has an inhibitory capacity for at least one selected from the group consisting of DYRK1A, DYRK1B, and DYRK2. In another one or more embodiments, the compound with a DYRK inhibitory capacity has an inhibitory capacity for at least DYRK1A.

In one or more embodiments, a compound with a CLK inhibitory capacity means that the compound has an inhibitory capacity for at least one kinase that belongs to the CLK family. In another one or more embodiments, a compound with a CLK inhibitory capacity means that the compound has activity to inhibit at least one phosphorylation activity of a kinase that belongs to the CLK family. In one or more embodiments, the compound with a CLK inhibitory capacity has an inhibitory capacity for at least one selected from the group consisting of CLK1, CLK2, CLK3, and CLK4.

In the present disclosure, the compound with an inhibitory capacity for a kinase is defined as follows. In one or more embodiments, when the compound is added to at least one of known in vitro and in vivo assay systems for studying the inhibition of protein phosphorylation activity, it is possible to inhibit the protein phosphorylation activity to, e.g., 60% or less, preferably 50% or less, more preferably 40% or less, even more preferably 30% or less, further preferably 20% or less, and particularly preferably 10% or less, compared to the control in which the compound is not added. In one or more embodiments, the amount of the compound added to the assay system is 0.01 to 10 μM. In one or more embodiments, the assay of the inhibition of protein phosphorylation activity may be in vitro and/or in vivo assay disclosed in WO 2010/010797.

In one or more embodiments, the present disclosure is based on the findings that the compound having DYRK inhibitory activity can activate neurogenesis or the growth of neurons. Therefore, in one aspect, the present disclosure relates to a composition for activating neurogenesis or the growth of neurons. The composition contains as an active ingredient a compound with a DYRK inhibitory capacity or a prodrug thereof or a pharmaceutically acceptable salt thereof.

Although the details of the mechanism of the activation of neurogenesis or the growth of neurons by the composition of the present disclosure are not clear, the mechanism can be estimated as follows. The DYRK is considered to phosphorylate cyclin D1, which regulates the cell proliferation positively, so that the cyclin D1 is directed to a decomposition path. Since the compound having DYRK inhibitory activity acts to suppress the decomposition of the cyclin D1, the amount of the cyclin D1 is increased, and the cell proliferation is promoted. However, the present disclosure should not be limited to this mechanism.

In one or more embodiments, the active ingredient of the composition of this aspect has a CLK inhibitory capacity in addition to the DYRK inhibitory capacity.

[Activation of Neurogenesis]

In one or more embodiments, the composition of this aspect has the effect of the activation of neurogenesis. In one or more embodiments, “neurogenesis” in the present disclosure means division and growth of neural stem cells, production of neural precursor cells, differentiation and maturation of the produced neural precursor cells into neurons, or a combination of them in living organisms or adults. The living organisms or adults include, e.g., mammals, humans, and mammals other than humans. The “neural stem cells” in the present disclosure are present in the brain and spinal cord, and produce precursor cells having the ability to differentiate into neurons or glia cells. In one or more embodiments, the “activation of neurogenesis” in the present disclosure means division and growth of neural stem cells, production of neural precursor cells, differentiation and maturation of the produced neural precursor cells into neurons, or enhancement of the combination of them in living organisms or adults. In one or more embodiments, the composition of this aspect is a pharmaceutical composition.

In one or more embodiments, the composition or pharmaceutical composition of this aspect can activate neurogenesis, and thus have the effects of preventing, improving, inhibiting the development of, and/or treating diseases or disorders of the central and/or peripheral nervous systems when they are administered to a subject. In one or more embodiments, the diseases or disorders of the central and/or peripheral nervous systems are caused by hippocampal atrophy, and may include, e.g., intellectual disability, learning disability, mood disorder, PTSD and anxiety disorder, organic mental disorder including symptom disorder, and substance-related disorder (particularly alcohol-related disorder and stimulants). In one or more embodiments, examples of the organic mental disorder include the following: injury; infection; angiopathy; Alzheimer's disease or other dementias caused by degeneration and metabolic disorder; Parkinson's disease; Huntington's disease; traumatic neurosis; mild cognitive impairment (MCI); psychological symptoms after brain infarction (such as depression and dysmnesia); ischemic hippocampal damage (due to short-time cardiac arrest); spinal cord injury; open or penetrating head injury caused by surgery; and closed head injury caused by, e.g., damage to the head region.

Therefore, in one or more embodiments, the present disclosure relates to a pharmaceutical composition for preventing, improving, inhibiting the development of, and/or treating diseases or disorders of the central and/or peripheral nervous systems. The pharmaceutical composition contains as an active ingredient a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof.

In one or more embodiments, the present disclosure relates to a method for activating neurogenesis, which includes administering the pharmaceutical composition of the present disclosure to a subject. In one or more embodiments, examples of the subject include mammals, humans, and mammals other than humans. In another one or more embodiments, the present disclosure relates to a method for preventing, improving, inhibiting the development of, and/or treating diseases or disorders of the central and/or peripheral nervous systems, which includes administering the pharmaceutical composition of the present disclosure to a subject. In one or more embodiments, the present disclosure relates to the use of the pharmaceutical composition of the present disclosure in the method for activating neurogenesis of the present disclosure. In another one or more embodiments, the present disclosure relates to the use of the pharmaceutical composition of the present disclosure in the method for preventing, improving, inhibiting the development of, and/or treating diseases or disorders of the central and/or peripheral nervous systems of the present disclosure. Moreover, in one or more embodiments, the present disclosure relates to the use of a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof in production of the pharmaceutical composition for activating neurogenesis of the present disclosure. In one or more embodiments, the present disclosure relates to the use of a compound with a DYRK inhibitory capacity or a prodrug thereof or a pharmaceutically acceptable salt thereof or a compound with a DYRK inhibitory capacity and a CLK inhibitory capacity or a prodrug thereof or a pharmaceutically acceptable salt thereof in production of the pharmaceutical composition for preventing, improving, inhibiting the development of, and/or treating diseases or disorders of the central and/or peripheral nervous systems of the present disclosure.

The present disclosure may relate to one or more embodiments below.

[a1]A composition for activating neurogenesis, containing as an active ingredient a compound with a DYRK inhibitory capacity or a prodrug thereof or a pharmaceutically acceptable salt thereof.

[a2] The composition according to [a1], wherein the compound or the prodrug thereof or the pharmaceutically acceptable salt thereof as the active ingredient further has a CLK inhibitory capacity.

[a3] The composition according to [a1] or [a2], wherein the composition is a pharmaceutical composition.

[a4]A pharmaceutical composition for preventing, improving, inhibiting the development of and/or treating diseases or disorders of the central and/or peripheral nervous systems, the pharmaceutical composition containing as an active ingredient a compound with a DYRK inhibitory capacity or a prodrug thereof or a pharmaceutically acceptable salt thereof or a compound with a DYRK inhibitory capacity and a CLK inhibitory capacity or a prodrug thereof or a pharmaceutically acceptable salt thereof.

[a5]A method for activating neurogenesis of a subject, including:

administering a pharmaceutical composition to the subject,

the pharmaceutical composition containing as an active ingredient a compound with a DYRK inhibitory capacity or a prodrug thereof or a pharmaceutically acceptable salt thereof or a compound with a DYRK inhibitory capacity and a CLK inhibitory capacity or a prodrug thereof or a pharmaceutically acceptable salt thereof.

[a6]A method for preventing, improving, inhibiting the development of, and/or treating diseases or disorders of the central and/or peripheral nervous systems, including:

administering a pharmaceutical composition to a subject,

the pharmaceutical composition containing as an active ingredient a compound with a DYRK inhibitory capacity or a prodrug thereof or a pharmaceutically acceptable salt thereof or a compound with a DYRK inhibitory capacity and a CLK inhibitory capacity or a prodrug thereof or a pharmaceutically acceptable salt thereof.

[a7] Use of a pharmaceutical composition in activation of neurogenesis,

the pharmaceutical composition containing as an active ingredient a compound with a DYRK inhibitory capacity or a prodrug thereof or a pharmaceutically acceptable salt thereof; or a compound with a DYRK inhibitory capacity and a CLK inhibitory capacity or a prodrug thereof or a pharmaceutically acceptable salt thereof.

[a8] Use of a pharmaceutical composition in prevention, improvement,

inhibition of the development, and/or treatment of diseases or disorders of the central and/or peripheral nervous systems,

the pharmaceutical composition containing as an active ingredient a compound with a DYRK inhibitory capacity or a prodrug thereof or a pharmaceutically acceptable salt thereof; or a compound with a DYRK inhibitory capacity and a CLK inhibitory capacity or a prodrug thereof or a pharmaceutically acceptable salt thereof.

[a9] Use of a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof as an active ingredient in production of a pharmaceutical composition for activating neurogenesis.

[a10] Use of a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof as an active ingredient in production of a pharmaceutical composition for preventing, improving, inhibiting the development of, and/or treating diseases or disorders of the central and/or peripheral nervous systems.

[Activation of Growth of Neurons]

In one or more embodiment, the compound with a DYRK inhibitory capacity or the prodrug thereof or the pharmaceutically acceptable salt thereof, or the compound with a DYRK inhibitory capacity and a CLK inhibitory capacity or the prodrug thereof or the pharmaceutically acceptable salt thereof has the effect of the activation of the growth of neurons. In one or more embodiments, “neurons” in the present disclosure include neural stem cells. As described above, the “neural stem cells” in the present disclosure are present in the brain and spinal cord, and produce precursor cells having the ability to differentiate into neurons or glia cells. In one or more embodiments, the “growth of neurons” in the present disclosure means the growth of neurons or neural stem cells (also referred to as “neural (stem) cells” in the following). In one or more embodiments, the “growth of neural (stem) cells” in the present disclosure means the growth of neural (stem) cells in vitro, in vivo, or ex vivo. Alternatively, in one or more embodiments, it means the growth of cultured neural stem cells. In one or more embodiments, the “cultured neural stem cells” in the present disclosure means a mass of neural stem cells that have been isolated from living organisms and cultured. In one or more embodiments, the “activation of the growth of neurons” in the present disclosure means that the growth of neural (stem) cells is activated. In another one or more embodiments, it also means that the production of neural precursor cells is promoted. In one or more embodiments, the “activation of the growth of neurons” means the activation of the growth of neural (stem) cells in vitro, in vivo, or ex vivo. In another one or more embodiments, it also means the activation of the growth of cultured neural stem cells.

Therefore, in one aspect, the present disclosure relates to a composition for activating the growth of neurons. The composition contains as an active ingredient a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof. The composition of this aspect may be a pharmaceutical composition.

In one or more embodiments, the composition of this aspect can enhance the growth of cultured neural stem cells, and thus is expected to promote the growth of neural stem cells that are present in the brain and spinal cord of living organisms.

Therefore, in one or more embodiments, the present disclosure relates to a composition for activating cultured neural stem cells. The composition contains as an active ingredient a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof.

In one or more embodiments, the present disclosure relates to a method for growing neural (stem) cells, which includes culturing neural (stem) cells in a culture medium containing the composition of the present disclosure. In one or more embodiments, the present disclosure relates to a method for preparing neural (stem) cells, which includes culturing neural (stem) cells in a culture medium containing the composition of the present disclosure. Moreover, in one or more embodiments, the present disclosure relates to the use of the composition of the present disclosure in the method for growing neural (stem) cells of the present disclosure. In one or more embodiments, the present disclosure relates to the use of the composition of the present disclosure in the method for preparing neural (stem) cells of the present disclosure.

The present disclosure may relate to one or more embodiments below.

[b1]A composition for activating the growth of neurons, containing as an active ingredient a compound with a DYRK inhibitory capacity or a prodrug thereof or a pharmaceutically acceptable salt thereof.

[b2] The composition according to [b1], wherein the compound or the prodrug thereof or the pharmaceutically acceptable salt thereof as the active ingredient further has a CLK inhibitory capacity.

[b3]A composition for activating the growth of neural (stem) cells, containing as an active ingredient a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof.

[b4] The composition according to any one of [b1] to [b3], wherein the composition is a pharmaceutical composition.

[b5]A method for activating the growth of neurons, including:

culturing neural (stem) cells in a culture medium containing a composition that contains as an active ingredient a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof.

[b6]A method for preparing neural (stem) cells, including:

culturing neural (stem) cells in a culture medium containing a composition that contains as an active ingredient a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof.

[b7] Use of a composition in activation of the growth of neurons,

the composition containing as an active ingredient a compound with a DYRK inhibitory capacity or a prodrug thereof or a pharmaceutically acceptable salt thereof or a compound with a DYRK inhibitory capacity and a CLK inhibitory capacity or a prodrug thereof or a pharmaceutically acceptable salt thereof.

[b8] Use of a composition in preparation of neural (stem) cells,

the composition containing as an active ingredient a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof.

[b9] Use of a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof as an active ingredient in production of a composition for activating the growth of neurons.

[b10] Use of a compound with a DYRK inhibitory capacity or a prodrug thereof or a pharmaceutically acceptable salt thereof, or a compound with a DYRK inhibitory capacity and a CLK inhibitory capacity or a prodrug thereof or a pharmaceutically acceptable salt thereof as an active ingredient in production of a composition for preparing neural (stem) cells.

[Compound Expressed by General Formula (I)]

In one or more embodiments, the present disclosure relates to a compound expressed by the following general formula (I) or a prodrug thereof or a pharmaceutically acceptable salt thereof.

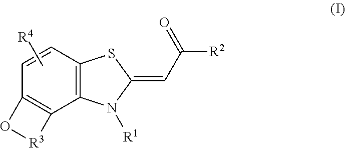

(where, in the general formula (I), R¹ and R² each independently represent a hydrogen atom or a C₁₋₆ hydrocarbon chain,

R³ represents

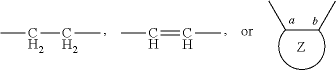

where Z and atoms marked with a and b form a ring selected from the group consisting of one benzene ring, one heteroaromatic ring, an aromatic ring in which one or more benzene rings are condensed, a heteroaromatic ring in which one or more heteroaromatic rings are condensed, a mixed condensed polycyclic ring in which one or more benzene rings are condensed with one or more heteroaromatic rings, and a cyclic aliphatic, and the ring may have at least one substituent that is a hydrogen atom, a halogen atom, or a C₁₋₆ alkyl group, and

R⁴ represents a hydrogen atom, a halogen atom, or a C₁₋₆ alkyl group.)

In one or more embodiments, the “prodrug” in the present disclosure may be a compound that is easily hydrolyzed in a living organism to regenerate the compound expressed by the general formula (I). If a compound has, e.g., a carboxyl group, the prodrug of the compound may be a compound in which the carboxyl group is converted to an alkoxycarbonyl group, a compound in which the carboxyl group is converted to an alkylthiocarbonyl group, or a compound in which the carboxyl group is converted to an alkylaminocarbonyl group. Moreover, if a compound has, e.g., an amino group, the prodrug of the compound may be a compound in which the amino group is substituted with an alkanoyl group to form an alkanoylamino group, a compound in which the amino group is substituted with an alkoxycarbonyl group to form an alkoxycarbonylamino group, a compound in which the amino group is converted to an acyloxymethylamino group, or a compound in which the amino group is converted to hydroxylamine. Further, if a compound has, e.g., a hydroxyl group, the prodrug of the compound may be a compound in which the hydroxyl group is substituted with the acyl group to form an acyloxy group, a compound in which the hydroxyl group is converted to a phosphoric ester, or a compound in which the hydroxyl group is converted to an acyloxymethyloxy group. The alkyl portion of the group used for the conversion to the prodrug may be an alkyl group, as will be described later. The alkyl group may be substituted (e.g., with an alkoxy group having 1 to 6 carbon atoms). In one or more embodiments, e.g., when the prodrug is a compound obtained by converting the carboxyl group to an alkoxycarbonyl group, the compound may include lower alkoxycarbonyl (e.g., having 1 to 6 carbon atoms) such as methoxycarbonyl and ethoxycarbonyl, or lower alkoxycarbonyl (e.g., having 1 to 6 carbon atoms) substituted with an alkoxy group such as methoxymethoxycarbonyl, ethoxymethoxycarbonyl, 2-methoxyethoxycarbonyl, 2-methoxyethoxymethoxycarbonyl, and pivaloyloxymethoxycarbonyl.

The “C₁₋₆ hydrocarbon chain” in the present disclosure refers to a monovalent group induced by removing any one of hydrogen atoms from an aliphatic hydrocarbon having 1 to 6 carbon atoms. In one or more embodiments, the hydrocarbon chain may have a linear, branched, or cyclic structure and may be an alkyl group, an alkenyl group, a phenyl group, or a cycloalkyl group. In one or more embodiments, examples of the “C₁₋₆ alkyl group” in the present disclosure include the following: a methyl group; an ethyl group; a 1-propyl group; a 2-propyl group; a 2-methyl-1-propyl group; a 2-methyl-2-propyl group; a 1-butyl group; a 2-butyl group; a 1-pentyl group; a 2-pentyl group; a 3-pentyl group; a 2-methyl-1-butyl group; a 3-methyl-1-butyl group; a 2-methyl-2-butyl group; a 3-methyl-2-butyl group; a 2,2-dimethyl-1-propyl group; a 1-hexyl group; a 2-hexyl group; a 3-hexyl group; a 2-methyl-1-pentyl group; a 3-methyl-1-pentyl group; a 4-methyl-1-pentyl group; a 2-methyl-2-pentyl group; a 3-methyl-2-pentyl group; a 4-methyl-2-pentyl group; a 2-methyl-3-pentyl group; a 3-methyl-3-pentyl group; a 2,3-dimethyl-1-butyl group; a 3,3-dimethyl-1-butyl group; a 2,2-dimethyl-1-butyl group; a 2-ethyl-1-butyl group; a 3,3-dimethyl-2-butyl group; and a 2,3-dimethyl-2-butyl group.

The “heterocyclic ring” in the present disclosure contains 1 to 2 hetero atoms as ring member atoms and may have a double bond. The heterocyclic ring means a non-aromatic ring or an aromatic ring. The “heteroaromatic ring” in the present disclosure means an aromatic heterocyclic ring. The “hetero atom” in the present disclosure means a sulfur atom, an oxygen atom, or a nitrogen atom.

The “cyclic aliphatic” in the present disclosure means an aliphatic having a cyclic structure. The group of the cyclic aliphatic may be, e.g., either a cyclic aliphatic group having 3 to 10 carbon atoms or a cyclic aliphatic group having a condensed ring structure of a plurality of rings. Specific examples of the cyclic aliphatic group include a cycloalkyl group having 3 to 10 carbon atoms, a cyclic ether group, a decahydronaphthyl group, and an adamantly group. Specific examples of the cyclic aliphatic group having 3 to 10 carbon atoms include a cyclopropyl group, a cyclobutyl group, a cyclopentyl group, a cyclohexyl group, and a cycloheptyl group.

The “pharmaceutically acceptable salt” in the present disclosure includes a pharmacologically and/or medically acceptable salt, and may be, e.g., an inorganic acid salt, an organic acid salt, an inorganic base salt, an organic base salt, or an acidic or basic amino acid salt.

Preferred examples of the inorganic acid salt include the following: hydrochloride; hydrobromate; sulfate; nitrate; and phosphate. Preferred examples of the organic acid salt include the following: acetate; succinate; fumarate; maleate; tartrate; citrate; lactate; stearate; benzoate; methanesulfonate; and p-toluenesulfonate.

Preferred examples of the inorganic base salt include the following: alkali metal salts such as sodium salt and potassium salt; alkaline-earth metal salts such as calcium salt and magnesium salt; aluminum salts; and ammonium salts. Preferred examples of the organic base salt include the following: diethylamine salt; diethanolamine salt; meglumine salt; and N,N′-dibenzylethylenediamine salt.

Preferred examples of the acidic amino acid salt include aspartate and glutamate. Preferred examples of the basic amino acid salt include arginine salt, lysine salt, and ornithine salt.

The “salt of the compound” in the present disclosure may include a hydrate that can be formed by allowing the compound to stand in the air so that it absorbs water. Moreover, the “salt of the compound” in the present disclosure may also include a solvate that can be formed by letting the compound absorb some type of solvent.

In one or more embodiments, R¹ of the general formula (I) represents a C₁₋₆ alkyl group. Moreover, in one or more embodiments, R¹ represents a methyl group, an ethyl group, or a propyl group. In one or more embodiments, R² of the general formula (I) represents a C₁₋₆ alkyl group. Moreover, in one or more embodiments, R² represents a methyl group. In one or more embodiments, R³ of the general formula (I) represents

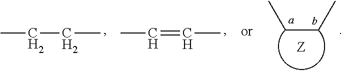

In one or more embodiments, R³ is —CH₂—CH₂— or —CH═CH—. In one or more embodiments, Z and atoms marked with a and b form one benzene ring. In one or more embodiments, R⁴ of the general formula (I) represents a hydrogen atom.

In one or more embodiments, the compound expressed by the general formula (I) is a compound expressed by

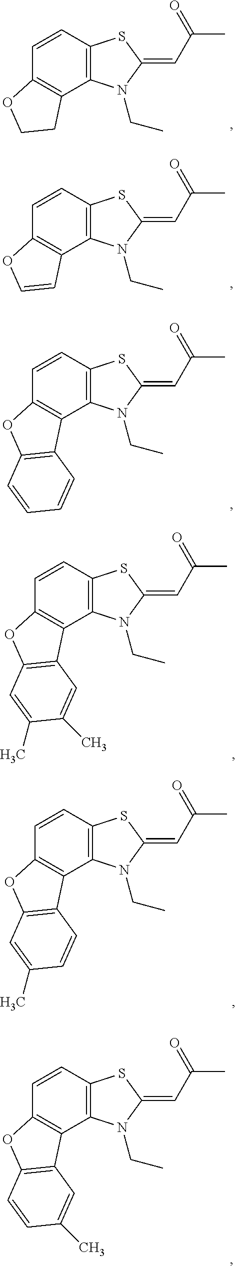

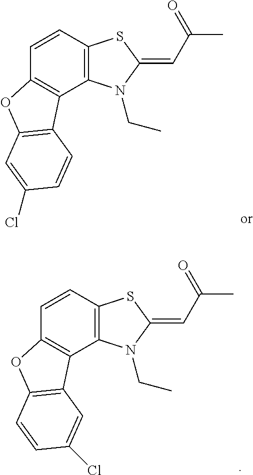

In one or more embodiments of the present disclosure, the compound expressed by the general formula (I) or the prodrug thereof or the pharmaceutically acceptable salt thereof has a DYRK inhibitory capacity. In one or more embodiments of the present disclosure, the compound expressed by the general formula (I) or the prodrug thereof or the pharmaceutically acceptable salt thereof has a DYRK inhibitory capacity and a CLK inhibitory capacity.

[Compound Expressed by General Formula (II)]

In one or more embodiments, the present disclosure relates to a compound expressed by the following general formula (II) or a prodrug thereof or a pharmaceutically acceptable salt thereof

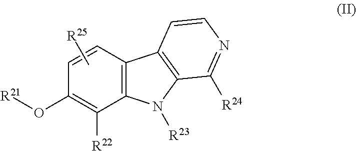

(where, in the general formula (II), R²¹ and R²³ each independently represent a hydrogen atom, a linear, branched, or cyclic C₁₋₆ alkyl group, a benzyl or heteroarylmethyl group, a substituted or unsubstituted aryl group, or a substituted or unsubstituted heteroaryl group,

R²² is selected from the group consisting of —R²⁶, —C≡C—R²⁶, —CH═CH—R²⁶, and —O—(CH₂)n-R²⁶, where n is 1 to 6, R²⁶ is selected from the group consisting of a hydrogen atom, a hydroxyl group, a C₁₋₈ alkyl group, —Si(R²⁷)₃, a substituted or unsubstituted phenyl group, a monocyclic heteroaromatic ring group, and a cyclic aliphatic group, R²⁷ represents a hydrogen atom, a C₁₋₆ alkyl group, a trihalomethyl group, or a hydroxyl group, and three elements represented by R²⁷ of —Si(R²⁷)₃ may differ from each other, alternatively R²² is bonded with R²¹ to form a ring, and —R²¹-R²²— is selected from the group consisting of —(CH₂)m-CH₂—, —CH═CH—, —(CH₂)m-O—, halogen-substituted —(CH₂)m-CH₂—, halogen-substituted —CH═CH—, and halogen-substituted —(CH₂)m-O—, where m is 1 to 6, and

R²⁴ and R²⁵ represent a hydrogen atom or a C₁₆ alkyl group.)

In one or more embodiments, examples of heteroaryl (including heteroaryl of the heteroarylmethyl group) of the general formula (II) include the following: a 5- to 6-membered monocyclic group containing 1 to 2 nitrogen atom(s); a 5- to 6-membered monocyclic group containing 1 to 2 nitrogen atom(s) and either 1 oxygen atom or 1 sulfur atom; a 5-membered monocyclic group containing 1 oxygen atom or 1 sulfur atom; and a bicyclic group that contains 1 to 4 nitrogen atom(s) and is formed by the condensation of a 6-membered ring and a 5- or 6-membered ring. In another one or more embodiments, examples of the heteroaryl include the following: 2-pyridyl; 3-pyridyl; 4-pyridyl; 2-thienyl, 3-thienyl, 3-oxadiazolyl, 2-imidazolyl, 2-thiazolyl, 3-isothiazolyl, 2-oxazolyl, 3-isoxazolyl, 2-furyl, 3-furyl, 3-pyrrolyl, 2-quinolyl, 8-quinolyl, 2-quinazolinyl, and 8-purinyl. Examples of the aryl group include an aryl group having 10 or less carbon atoms such as a phenyl group or a naphthyl group.

The number of substituents of the phenyl group, the monocyclic heteroaromatic ring group, the cyclic aliphatic group, the aryl group, and the heteroaryl group (including heteroaryl of the heteroarylmethyl group) of the general formula (II) may be one or more than one, and the substituents may be either the same or different. In one or more embodiments, examples of the substituent include the following: a halogen atom; a cyano group; a trifluoromethyl group; a nitro group; a hydroxyl group; a methylenedioxy group; a lower alkyl group; a lower alkoxy group; a benzyloxy group; a lower alkanoyloxy group; an amino group; a mono-lower alkylamino group; a di-lower alkylamino group; a carbamoyl group; a lower alkylaminocarbonyl group; di-lower alkylaminocarbonyl group; a carboxyl group; a lower alkoxycarbonyl group; a lower alkylthio group; a lower alkylsulfinyl group; a lower alkylsulfonyl group; a lower alkanoylamino group; and a lower alkylsulfonamide group. In one or more embodiments, the halogen atom may be, e.g., a fluorine atom, a chlorine atom, a bromine atom, or an iodine atom. In one or more embodiments, the lower alkyl may be the “C₁₋₆ alkyl group” as defined above.

In one or more embodiment, R²¹ of the general formula (II) represents a hydrogen atom or a C₁₋₃ alkyl group. In one or more embodiments, R²² of the general formula (II) represents —R²⁶ or —C≡C—R²⁶. In one or more embodiments, R²⁶ is selected from the group consisting of —Si(R²⁷)₃, a substituted or unsubstituted phenyl group, a monocyclic heteroaromatic ring group, and a cyclic aliphatic group. In one or more embodiments, R²⁷ represents a C₁₋₃ alkyl group. In one or more embodiments, R²³ of the general formula (II) represents a hydrogen atom or a C₁₋₆ alkyl group. In one or more embodiments, R²⁴ and R²⁵ of the general formula (II) represent a hydrogen atom or a C₁₋₃ alkyl group.

In one or more embodiments, the compound expressed by the general formula (II) does not contain harmine. In one or more embodiments, R²¹, R²², R²³, R²⁴, and R²⁵ of the general formula (II) are not combined into harmine (i.e., the compound does not have a combination of a methyl group for R²¹, hydrogen atoms for R²² and R²³, a methyl group for R²⁴, and a hydrogen atom for R²⁵).

In one or more embodiments, the compound expressed by the general formula (II) or the pharmaceutically acceptable salt thereof is a compound expressed by

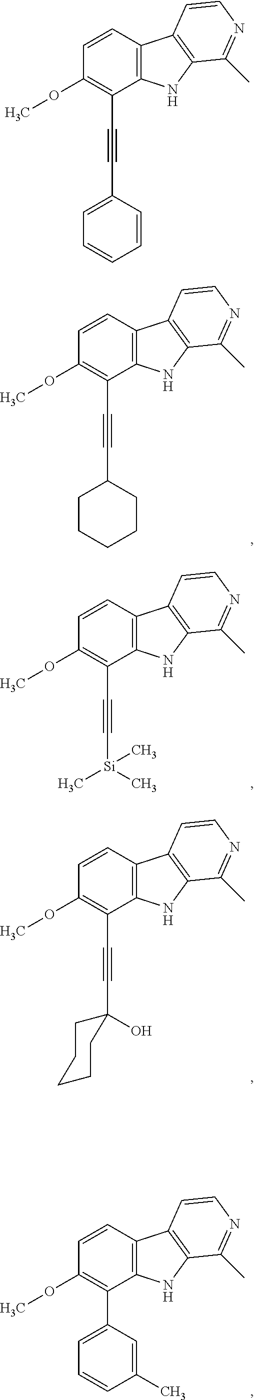

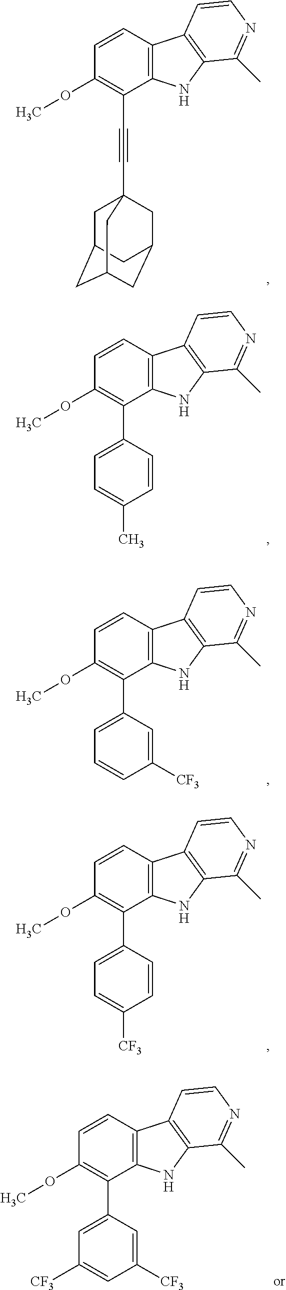

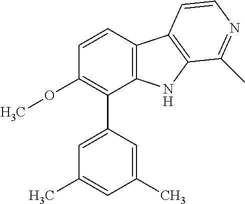

or a pharmaceutically acceptable salt thereof.

In one or more embodiments of the present disclosure, the compound expressed by the general formula (II) or the prodrug thereof or the pharmaceutically acceptable salt thereof has a DYRK inhibitory capacity. In one or more embodiments of the present disclosure, the compound expressed by the general formula (II) or the prodrug thereof or the pharmaceutically acceptable salt thereof has a DYRK inhibitory capacity and a CLK inhibitory capacity.

[Activation of Neurogenesis]

In one or more embodiments, the compound expressed by the general formula (I) or (II) or the prodrug thereof or the pharmaceutically acceptable salt thereof has the effect of the activation of neurogenesis. As described above, in one or more embodiments, the “activation of neurogenesis” in the present disclosure means division and growth of neural stem cells, production of neural precursor cells, differentiation and maturation of the produced neural precursor cells into neurons, or enhancement of the combination of them in living organisms or adults. In one or more embodiments, the composition of this aspect is a pharmaceutical composition.

Therefore, in one aspect, the present disclosure relates to a pharmaceutical composition for activating neurogenesis. The pharmaceutical composition contains as an active ingredient a compound expressed by the general formula (I) or (II) or a prodrug thereof or a pharmaceutically acceptable salt thereof. In one or more embodiments, the compound expressed by the general formula (I) or (II) or the prodrug thereof or the pharmaceutically acceptable salt thereof exhibits intracerebral transferability and oral absorbability. Because of these properties, neurogenesis can be activated more effectively.

In one or more embodiments, the composition or pharmaceutical composition of this aspect can activate neurogenesis, and thus have the effects of preventing, improving, inhibiting the development of, and/or treating diseases or disorders of the central and/or peripheral nervous systems when they are administered to a subject. In one or more embodiments, the diseases or disorders of the central and/or peripheral nervous systems are caused by hippocampal atrophy, and may include, e.g., intellectual disability, learning disability, mood disorder, PTSD and anxiety disorder, organic mental disorder including symptom disorder, and substance-related disorder (particularly alcohol-related disorder and stimulants). In one or more embodiments, examples of the organic mental disorder include the following: injury; infection; angiopathy; Alzheimer's disease or other dementias caused by degeneration and metabolic disorder; Parkinson's disease; Huntington's disease; traumatic neurosis; mild cognitive impairment (MCI); psychological symptoms after brain infarction (such as depression and dysmnesia); ischemic hippocampal damage (due to short-time cardiac arrest); spinal cord injury; open or penetrating head injury caused by surgery; and closed head injury caused by, e.g., damage to the head region.

Therefore, in one or more embodiments, the present disclosure relates to a pharmaceutical composition for preventing, improving, inhibiting the development of, and/or treating diseases or disorders of the central and/or peripheral nervous systems. The pharmaceutical composition contains as an active ingredient a compound expressed by the general formula (I) or (II) or a prodrug thereof or a pharmaceutically acceptable salt thereof.

In one or more embodiments, the present disclosure relates to a method for activating neurogenesis, which includes administering the pharmaceutical composition of the present disclosure to a subject. In one or more embodiments, examples of the subject include mammals, humans, and mammals other than humans. In another one or more embodiments, the present disclosure relates to a method for preventing, improving, inhibiting the development of, and/or treating diseases or disorders of the central and/or peripheral nervous systems, which includes administering the pharmaceutical composition of the present disclosure to a subject. In one or more embodiments, the present disclosure relates to the use of the pharmaceutical composition of the present disclosure in the method for activating neurogenesis of the present disclosure. In another one or more embodiments, the present disclosure relates to the use of the pharmaceutical composition of the present disclosure in the method for preventing, improving, inhibiting the development of, and/or treating diseases or disorders of the central and/or peripheral nervous systems of the present disclosure. Moreover, in one or more embodiments, the present disclosure relates to the use of a compound expressed by the general formula (I) or (II) or a prodrug thereof or a pharmaceutically acceptable salt thereof in production of the pharmaceutical composition for activating neurogenesis of the present disclosure. In one or more embodiments, the present disclosure relates to the use of a compound expressed by the general formula (I) or (II) or a prodrug thereof or a pharmaceutically acceptable salt thereof in production of the pharmaceutical composition for preventing, improving, inhibiting the development of, and/or treating diseases or disorders of the central and/or peripheral nervous systems of the present disclosure.

The present disclosure may relate to one or more embodiments below.

[c1]A composition for activating neurogenesis, containing as an active ingredient a compound expressed by the general formula (I) or (II) or a prodrug thereof or a pharmaceutically acceptable salt thereof.

[c2] The composition according to [c1], wherein the compound expressed by the general formula (I) is a compound expressed by

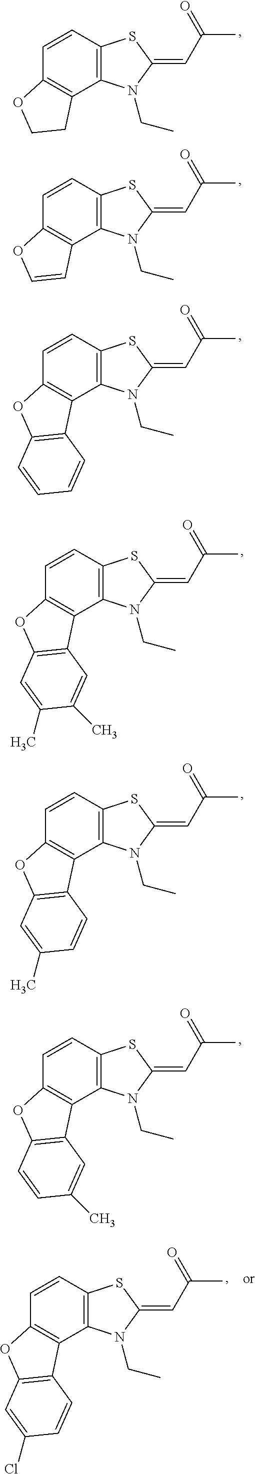

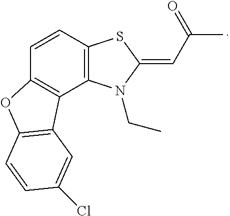

[c3] The composition according to [c1], wherein the compound expressed by the general formula (II) is a compound expressed by

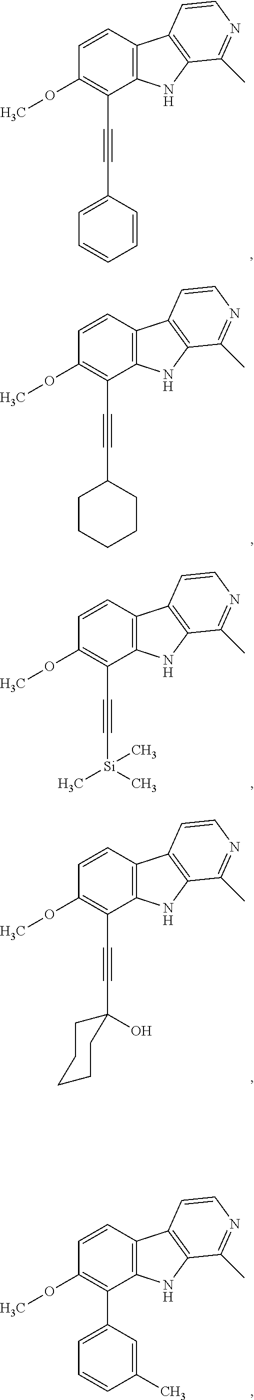

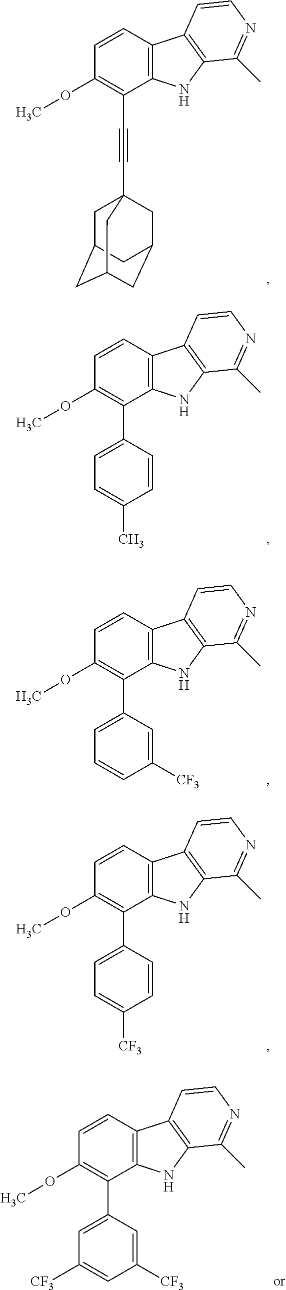

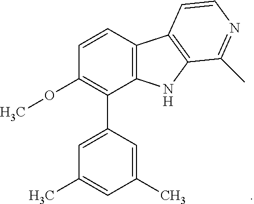

[c4] The composition according to any one of [c1] to [c3], wherein the compound expressed by the general formula (I) or (II) or the prodrug thereof or the pharmaceutically acceptable salt thereof has a DYRK inhibitory capacity.

[c5] The composition according to any one of [c1] to [c4], wherein the compound expressed by the general formula (I) or (II) or the prodrug thereof or the pharmaceutically acceptable salt thereof further has a CLK inhibitory capacity.

[c6] The composition according to any one of [c1] to [c5], wherein the composition is a pharmaceutical composition.

[c7]A pharmaceutical composition for preventing, improving, inhibiting the development of, and/or treating diseases or disorders of the central and/or peripheral nervous systems, the pharmaceutical composition containing as an active ingredient the compound expressed by the general formula (I) or (II) or the prodrug thereof or the pharmaceutically acceptable salt thereof according to any one of [c1] to [c5].

[c8]A method for activating neurogenesis of a subject, including:

administering a pharmaceutical composition to the subject,

the pharmaceutical composition containing as an active ingredient the compound expressed by the general formula (I) or (II) or the prodrug thereof or the pharmaceutically acceptable salt thereof according to any one of [c1] to [c5].

[c9]A method for preventing, improving, inhibiting the development of, and/or treating diseases or disorders of the central and/or peripheral nervous systems, including:

administering a pharmaceutical composition to a subject,

the pharmaceutical composition containing as an active ingredient the compound expressed by the general formula (I) or (II) or the prodrug thereof or the pharmaceutically acceptable salt thereof according to any one of [c1] to [c5].

[c10] Use of a pharmaceutical composition in activation of neurogenesis,

the pharmaceutical composition containing as an active ingredient the compound expressed by the general formula (I) or (II) or the prodrug thereof or the pharmaceutically acceptable salt thereof according to any one of [c1] to [c5].

[c11] Use of a pharmaceutical composition in prevention, improvement, inhibition of the development, and/or treatment of diseases or disorders of the central and/or peripheral nervous systems,

the pharmaceutical composition containing as an active ingredient the compound expressed by the general formula (I) or (II) or the prodrug thereof or the pharmaceutically acceptable salt thereof according to any one of [c1] to [c5].

[c12] Use of the compound expressed by the general formula (I) or (II) or the prodrug thereof or the pharmaceutically acceptable salt thereof according to any one of [c1] to [c5] as an active ingredient in production of a pharmaceutical composition for activating neurogenesis.

[c13] Use of the compound expressed by the general formula (I) or (II) or the prodrug thereof or the pharmaceutically acceptable salt thereof according to any one of [c1] to [c5] as an active ingredient in production of a pharmaceutical composition for preventing, improving, inhibiting the development of, and/or treating diseases or disorders of the central and/or peripheral nervous systems.

In one or more embodiments, the “pharmaceutical composition” of the present disclosure may have a dosage form suitable for administration by using the known formulation technology. Specifically, the pharmaceutical composition can be administered orally in dosage forms (but not limited to) such as tablets, capsules, granules, powder, pills, troche, syrups, and liquid formulations. Alternatively, the pharmaceutical composition can be administered parenterally in dosage forms (but not limited to) such as injection, liquid formulations, aerosol, suppositories, patches, cataplasm, lotions, liniments, ointments, and eye drops. These formulations can be produced by a known method using additives (but not limited to) such as excipients, lubricants, binders, disintegrators, stabilizers, corrigents, and diluents.

Examples of the excipient include (but not limited to) the following: starches such as starch, potato starch, and corn starch; lactose; crystalline cellulose; and calcium hydrogen phosphate. Examples of the coating agent include (but not limited to) the following: ethyl cellulose; hydroxypropyl cellulose; hydroxypropyl methylcellulose; shellac; talc; carnauba wax; and paraffin. Examples of the binder include (but not limited to) the following: polyvinyl pyrrolidone; macrogol; and the compounds similar to those given as examples of the excipient. Examples of the disintegrator include (but not limited to) the following: the compounds similar to those given as examples of the excipient; and chemically modified starches and celluloses such as croscarmellose sodium, sodium carboxymethyl starch, and cross-linked polyvinylpyrrolidone. Examples of the stabilizer include (but not limited to) the following: parahydroxybenzoic acid esters such as methylparaben and propylparaben; alcohols such as chlorobutanol, benzyl alcohol, and phenylethyl alcohol; benzalkonium chloride; phenols such as phenol and cresol; thimerosal; dehydroacetic acid; and sorbic acid. Examples of the corrigent include (but not limited to) commonly used sweeteners, acidulants, and flavors.

The preparation of a liquid formulation may use (but not limited to) ethanol, phenol, chlorocresol, purified water, or distilled water as a solvent, and may also use a surface-active agent or an emulsifying agent as needed. Examples of the surface-active agent or the emulsifying agent include (but not limited to) polysorbate 80, polyoxyl 40 stearate, and lauromacrogol.

The method for using the pharmaceutical composition of the present disclosure may differ depending on symptoms, ages, administration methods, etc. The method allows the pharmaceutical composition to be intermittently or continuously administered (but not limited to) orally, endermically, submucosally, subcutaneously, intramuscularly, intravascularly, intracerebrally, or intraperitoneally so that the concentration of the compound (active ingredient) expressed by the general formula (I) or (II) in the body is in the range of 100 nM to 1 mM. In a non-limiting embodiment, for oral administration, the pharmaceutical composition may be administered to a subject (e.g., an adult human) in a dosage of 0.01 mg (preferably 0.1 mg) to 2000 mg (preferably 500 mg and more preferably 100 mg), which is expressed in terms of the compound expressed by the general formula (I) or (II), once or several times a day based on the symptom. In a non-limiting embodiment, for intravenous administration, the pharmaceutical composition may be administered to a subject (e.g., an adult human) in a dosage of 0.001 mg (preferably 0.01 mg) to 500 mg (preferably 50 mg) once or several times a day based on the symptom.

[Activation of Growth of Neurons]

In one or more embodiments, the compound expressed by the general formula (I) or (II) or the prodrug thereof or the pharmaceutically acceptable salt thereof has the effect of the activation of the growth of neurons. As described above, in one or more embodiments, the “activation of the growth of neurons” in the present disclosure means that the growth of neural (stem) cells is activated. In another one or more embodiments, it also means that the production of neural precursor cells is promoted. As described above, in one or more embodiments, the “activation of the growth of neurons” means the activation of the growth of neural (stem) cells in vitro, in vivo, or ex vivo. In another one or more embodiments, it also means the activation of the growth of cultured neural stem cells.

Therefore, in one aspect, the present disclosure relates to a composition for activating the growth of neurons. The composition contains as an active ingredient a compound expressed by the general formula (I) or (II) or a prodrug thereof or a pharmaceutically acceptable salt thereof. The composition of this aspect may be a pharmaceutical composition.

In one or more embodiments, the composition of this aspect can enhance the growth of cultured neural stem cells, and thus is expected to promote the growth of neural stem cells that are present in the brain and spinal cord of living organisms.

Therefore, in one or more embodiments, the present disclosure relates to a composition for activating cultured neural stem cells. The composition contains as an active ingredient a compound expressed by the general formula (I) or (II) or a prodrug thereof or a pharmaceutically acceptable salt thereof.

In one or more embodiments, the present disclosure relates to a method for growing neural (stem) cells, which includes culturing neural (stem) cells in a culture medium containing the composition of the present disclosure. In one or more embodiments, the present disclosure relates to a method for preparing neural (stem) cells, which includes culturing neural (stem) cells in a culture medium containing the composition of the present disclosure. Moreover, in one or more embodiments, the present disclosure relates to the use of the composition of the present disclosure in the method for growing neural (stem) cells of the present disclosure. In one or more embodiments, the present disclosure relates to the use of the composition of the present disclosure in the method for preparing neural (stem) cells of the present disclosure.

The present disclosure may relate to one or more embodiments below.

[d1]A composition for activating the growth of neurons, containing as an active ingredient a compound expressed by the general formula (I) or (II) or a prodrug thereof or a pharmaceutically acceptable salt thereof.

[d2] The composition according to [d1], wherein the compound expressed by the general formula (I) is a compound expressed by

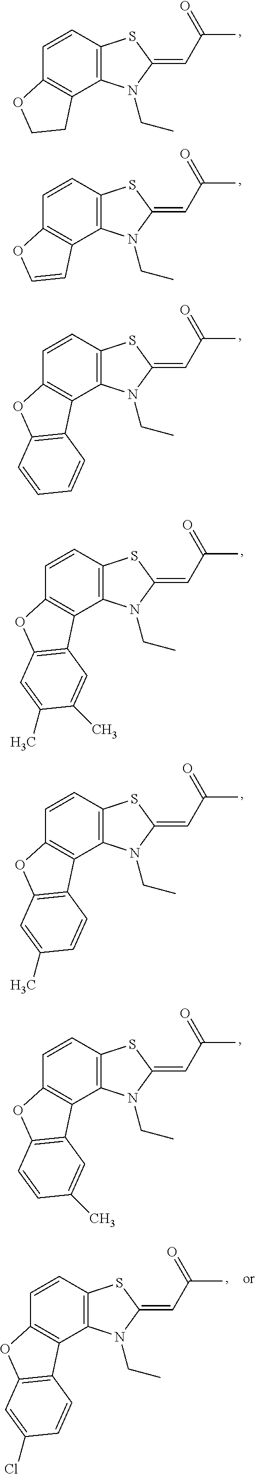

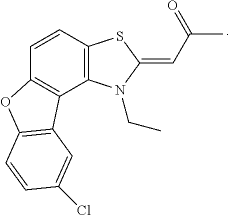

[d3] The composition according to [d1], wherein the compound expressed by the general formula (II) is a compound expressed by

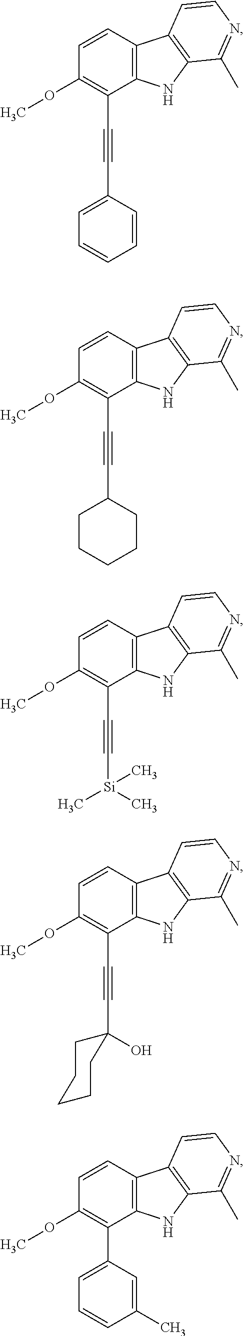

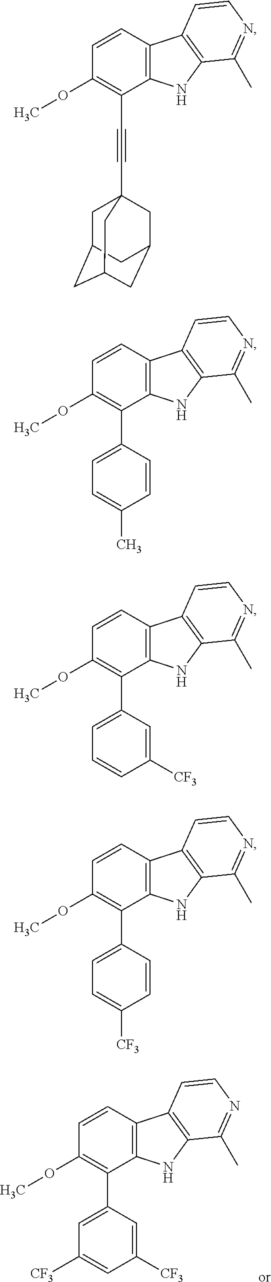

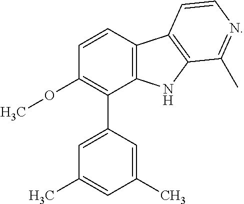

[d4] The composition according to any one of [d1] to [d3], wherein the compound expressed by the general formula (I) or (II) or the prodrug thereof or the pharmaceutically acceptable salt thereof has a DYRK inhibitory capacity.

[d5] The composition according to any one of [d1] to [d4], wherein the compound expressed by the general formula (I) or (II) or the prodrug thereof or the pharmaceutically acceptable salt thereof further has a CLK inhibitory capacity.

[d6] The composition according to any one of [d1] to [d5], wherein the composition is a pharmaceutical composition.

[d7]A composition for activating the growth of neural (stem) cells, containing as an active ingredient the compound expressed by the general formula (I) or (II) or the prodrug thereof or the pharmaceutically acceptable salt thereof according to any one of [d1] to [d5].

[d8]A method for activating the growth of neurons, including:

culturing neural (stem) cells in a culture medium containing a composition that contains as an active ingredient the compound expressed by the general formula (I) or (II) or the prodrug thereof or the pharmaceutically acceptable salt thereof according to any one of [d1] to [d5].

[d9]A method for preparing neural (stem) cells, including:

culturing neural (stem) cells in a culture medium containing a composition that contains as an active ingredient the compound expressed by the general formula (I) or (II) or the prodrug thereof or the pharmaceutically acceptable salt thereof according to any one of [d1] to [d5].

[d10] Use of a pharmaceutical composition in activation of the growth of neurons,

the composition containing as an active ingredient the compound expressed by the general formula (I) or (II) or the prodrug thereof or the pharmaceutically acceptable salt thereof according to any one of [d1] to [d5].

[d11] Use of a pharmaceutical composition in preparation of neural (stem) cells,

the composition containing as an active ingredient the compound expressed by the general formula (I) or (II) or the prodrug thereof or the pharmaceutically acceptable salt thereof according to any one of [d1] to [d5].

[d12] Use of the compound expressed by the general formula (I) or (II) or the prodrug thereof or the pharmaceutically acceptable salt thereof according to any one of [d1] to [d5] as an active ingredient in production of a pharmaceutical composition for activating the growth of neurons.

[d13] Use of the compound expressed by the general formula (I) or (II) or the prodrug thereof or the pharmaceutically acceptable salt thereof according to any one of [d1] to [d5] as an active ingredient in production of a pharmaceutical composition for preparing neural (stem) cells.

### EXAMPLES

Hereinafter, the present disclosure will be described in more detail by way of examples. These examples are for illustrative purposes only, and the present disclosure is not limited to the examples. All the documents cited in the present disclosure are incorporated herein by reference.

### Production Example 1

### Production of Compound 1

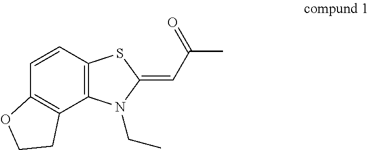 [SMILES: CCN1/C(=C/C(C)=O)Sc2ccc3c(c21)CCO3]

A compound 1 was produced in the following manner.

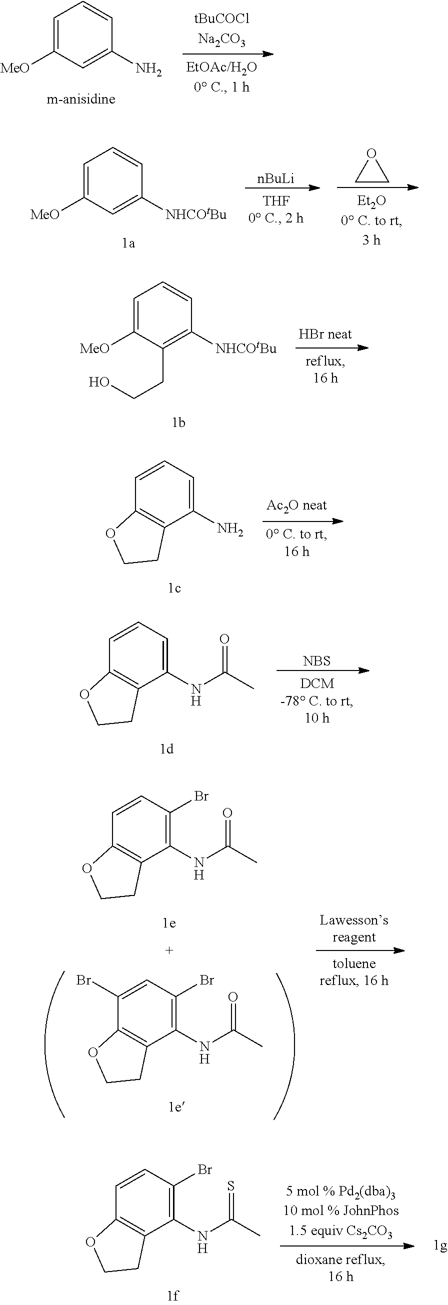

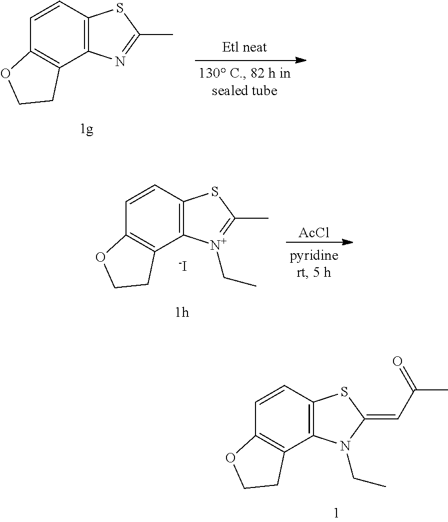

### Synthesis of N-(3-methoxyphenyl)pivalamide (1a)

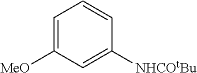

Under the argon atmosphere, pivaloyl chloride (25.0 mL, 205 mmol, commercial product) was slowly dropped at 0° C. into a mixed solution including m-anisidine (21.9 mL, 195 mmol, commercial product), ethyl acetate (EtOAc) (300 mL) of sodium carbonate monohydrate (62.0 g, 500 mmol, commercial product), and purified water (860 mL). After the mixture was stirred at 0° C. for 1 hour, the organic layer was separated and the aqueous layer was extracted with ethyl acetate (EtOAc). The combined organic layer was dried over sodium sulfate and filtered. The filtrate was concentrated under reduced pressure. The residue was recrystallized with ethyl acetate (EtOAc), and thus N-(3-methoxyphenyl)pivalamide (compound 1a) (40.2 g, 194 mmol, 99.5%) was obtained as a colorless solid.

TLC R_{f}=0.50 (n-hexane/EtOAc=6/1)

### Synthesis of N-[2-(2-hydroxyethyl)-3-methoxyphenyl]pivalamide (1b)

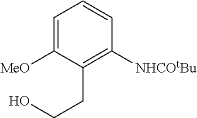

Under the argon atmosphere, n-butyllithium (nBuLi) (2.6 M in THF, 111 mL, 289 mmol, commercial product) was slowly dropped at 0° C. into a tetrahydrofuran (THF) (400 mL, dehydrated, commercial product) solution of the compound 1a (30.0 g, 145 mmol). After the mixture was stirred at 0° C. for 2 hours, ethylene oxide (1.3 M ether solution, 175 mL, 228 mmol, commercial product) was slowly added to the mixture and stirred at 0° C. for 1 hour. The temperature was raised to room temperature, and then the mixture was further stirred for 2 hours. The mixture was concentrated under reduced pressure, to which a saturated ammonium chloride aqueous solution (sat. NH₄Cl aq.) was added. Subsequently, the mixture was extracted with ethyl acetate (EtOAc) (100 mL×4). The combined organic layer was dried over sodium sulfate and filtered. The filtrate was concentrated under reduced pressure. The residue was recrystallized with ethyl acetate (EtOAc), and thus N-[2-(2-hydroxyethyl)-3-methoxyphenyl]pivalamide (compound 1b) (28.1 g, 112 mmol, 77.1%) was obtained as a colorless solid.

TLC R_{f}=0.40 (n-hexane/EtOAc=3/1)

### Synthesis of 4-amino-2,3-dihydrobenzofuran (1c)

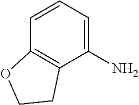

The compound 1b (4.10 g, 16.3 mmol) was dissolved in hydrobromic acid (HBr) (48% aqueous, 20.0 mL, commercial product), and the mixed solution was stirred by heating at 110° C. for 16 hours. After the mixed solution was allowed to cool to room temperature, sodium hydroxide granules were gradually added at 0° C. so that the pH was adjusted to about 9. Subsequently, the mixture was extracted with ethyl acetate (EtOAc) (50 mL×4). The combined organic layer was dried over sodium sulfate and filtered. The filtrate was concentrated under reduced pressure. The residue was purified by a medium-pressure column chromatography (Smart Flash EPCLC W-Prep 2XY system) (n-hexane/EtOAc=1/1), and thus 4-amino-2,3-dihydrobenzofuran (compound 1c) (1.49 g, 11.0 mmol, 67.6%) was obtained as a colorless solid.

TLC R_{f}=0.30 (n-hexane/EtOAc=1/1)

¹H NMR (400 MHz, CDCCl₃) δ (6.94 (dd, J=8.4, 8.4 Hz, 1H), 6.28 (dd, J=0.4, 7.6 Hz, 1H), 6.23 (dd, J=0.4, 7.6 Hz, 1H), 4.59 (t, J=8.4 Hz, 2H), 3.60 (brs, 2H), 3.02 (t, J=8.4 Hz, 2H)

### Synthesis of 4-acetylamino-2,3-dihydrobenzofuran (1d)

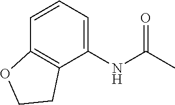

The compound 1c (2.00 g, 14.8 mmol) was dissolved in acetic anhydride (15.0 mL, commercial product), and the mixed solution was stirred at room temperature for 16 hours. After the reaction was completed, the mixture was concentrated under reduced pressure. The resultant brown solid was recrystallized with ethyl acetate (EtOAc), and thus 4-acetylamino-2,3-dihydrobenzofuran (compound 1d) (2.10 g, 11.9 mmol, 80.1%) was obtained as a colorless solid.

TLC R_{f}=0.15 (n-hexane/EtOAc=1/1)

¹H NMR (400 MHz, CDCl₃) δ 7.18 (d, J=6.4 Hz, 1H), 7.09 (t, J=6.4 Hz, 1H), 7.04 (brs, 1H), 6.62 (d, J=6.0 Hz, 1H), 4.59 (t, J=6.8 Hz, 2H), 3.13 (t, J=6.8 Hz, 2H), 2.18 (s, 3H)

### Synthesis of 4-acetylamino-5-bromo-2,3-dihydrobenzofuran (1e)

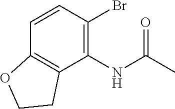

N-bromosuccinimide (2.31 g, 13.0 mmol, commercial product) was gradually added at −78° C. to a dichloromethane (50 ml, dehydrated, commercial product) solution of the compound 1d (2.10 g, 11.9 mmol), and the temperature was raised to room temperature for 10 hours. After the reaction was completed, the mixture was concentrated under reduced pressure. The residue was purified by a medium-pressure column chromatography (Smart Flash EPCLC W-Prep 2XY system) (n-hexane/EtOAc=1/1), and thus 4-acetylamino-5-bromo-2,3-dihydrobenzofuran (compound 1e) (1.76 g, 6.87 mmol, 57.8%) was obtained as a colorless solid. In this case, ¹H NMR analysis confirmed the by-production of a product (TLC R_{f}=0.15 (n-hexane/EtOAc=1/1)) that can be a dibromo body 1e′.

TLC R_{f}=0.25 (n-hexane/EtOAc=1/1)

¹H NMR (400 MHz, CDCl₃) δ 7.29 (d, J=8.4 Hz, 1H), 7.11 (brs, 1H), 6.58 (d, J=8.4 Hz, 1H), 4.60 (t, J=8.8 Hz, 2H), 3.22 (t, J=8.8 Hz, 2H), 2.23 (s, 3H)

### Synthesis of 5-bromo-4-thioacetylamino-2,3-dihydrobenzofuran (1f)

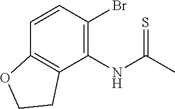

The compound 1e (1.76 g, 6.87 mmol) and a Lawesson's reagent (1.01 g, 2.50 mmol, commercial product) were dissolved in toluene (25 mL, dehydrated, commercial product). The mixture was heated to reflux for 16 hours. After the mixture was allowed to cool to room temperature, the mixture was concentrated under reduced pressure and purified by a medium-pressure column chromatography (Smart Flash EPCLC W-Prep 2XY system) (n-hexane/EtOAc=1/1), and thus 5-bromo-4-thioacetylamino-2,3-dihydrobenzofuran (compound 10 (1.86 g, 6.83 mmol, 99.5%) was obtained as a light brown solid.

TLC R_{f}=0.35 (n-hexane/EtOAc=1/1)

¹H NMR (400 MHz, CDCl₃) for a mixture of two rotamers (70:30) δ 8.85 (brs, 0.3H), 8.33 (brs, 0.7H), 7.41 (d, J=8.8 Hz, 0.3H), 7.36 (d, J=8.4 Hz, 0.7H), 6.73 (d, J=8.8 Hz, 0.3H), 6.69 (d, J=8.4 Hz, 0.7H), 4.69-4.59 (m, 2H), 3.19-3.27 (m, 2H), 2.76 (s, 2.1H), 2.36 (s, 0.9H)

### Synthesis of 2-methyl-7,8-dihydrobenzofuro[4,5-d]thiazole (1g)

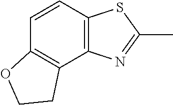

Under the argon atmosphere, trisdibenzylideneacetone (Pd₂(dba)₃) (237 mg, 0.259 mmol, commercial product), (2-biphenyl)-di-tert-butylphosphine (JohnPhos, 154 mg, 0.516 mmol, commercial product), and cesium carbonate (Cs₂CO₃) (2.50 g, 7.67 mmol, commercial product) were mixed with dioxane (30 mL, dehydrated, commercial product), and the mixture was stirred for 10 minutes. Then, a dioxane (20 mL, dehydrated, commercial product) solution of the compound if (1.40 g, 5.14 mmol) was added to this suspension and heated to reflux for 16 hours. After the mixture was allowed to cool to room temperature, the mixture was concentrated under reduced pressure. The residue was purified by a medium-pressure column chromatography (Smart Flash EPCLC W-Prep 2XY system), and thus 2-methyl-7,8-dihydrobenzofuro[4,5-d]thiazole (compound 1g) (780 mg, 4.08 mmol, 79.2%) was obtained as a light yellow solid.

TLC R_{f}=0.25 (n-hexane/EtOAc=1/1)

¹H NMR (400 MHz, CDCl₃) δ 7.45 (d, J=8.4 Hz, 1H), 6.82 (d, J=8.4 Hz, 1H), 4.63 (t, J=8.8 Hz, 2H), 3.51 (t, J=8.8 Hz, 2H), 2.75 (s, 3H)

### Synthesis of 1-ethyl-2-methyl-7,8-dihydrobenzofuro[4,5-d]thiazol-1-ium iodide (1h)

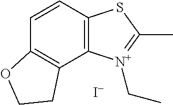

The compound 1g (182 mg, 0.952 mmol) was dissolved in iodoethane (EtI) (3.0 mL, commercial product), and the mixed solution was stirred by heating at 130° C. (i.e., the temperature of an aluminum heating block) for 82 hours. After the mixed solution was allowed to cool to room temperature, the iodoethane was distilled under reduced pressure, and the precipitated solid was filtered off with a Hirsch funnel. The solid was washed with ethyl acetate (3 mL×4) on the funnel and dried under reduced pressure, and thus 1-ethyl-2-methyl-7,8-dihydrobenzofuro[4,5-d]thiazol-1-ium iodide (compound 1h) (327 mg, 0.942 mmol, 98.9%) was obtained as a light yellow solid.

TLC a tailing spot R_{f}=0.25 (CH₂Cl₂/MeOH=5/1)

¹H NMR (400 MHz, CD₃OD) δ 8.00 (d, J=8.8 Hz, 1H), 3.17 (s, 3H), 7.27 (d, J=8.8 Hz, 1H), 4.82 (t, J=8.8 Hz, 2H), 4.76 (q, J=7.2 Hz, 2H), 3.86 (t, J=8.8 Hz, 2H), 1.59 (t, J=7.2 Hz, 3H)

### Synthesis of (Z)-1-[1-ethyl-7,8-dihydrobenzofuro[4,5-d]thiazol-2(1H)-ylidene]propan-2-one (compound 1)

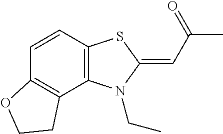 [SMILES: CCN1/C(=C/C(C)=O)Sc2ccc3c(c21)CCO3]

Under the argon atmosphere, acetyl chloride (61 μL, 0.86 mmol, commercial product) was added at 0° C. to a pyridine (4.0 mL, commercial product) solution of the compound 1h (150 mg, 0.432 mmol). The temperature was raised to room temperature, and then the mixture was stirred for 5 hours. After the reaction was completed, hydrochloric acid (0.25 M, 25 mL) was added to the mixture. Subsequently, the mixture was extracted with ethyl acetate (EtOAc) (3 mL×4). The combined organic layer was dried over sodium sulfate and filtered. The filtrate was concentrated under reduced pressure. The residue was purified by a medium-pressure column chromatography (Smart Flash EPCLC W-Prep 2XY system) (n-hexane/EtOAc=1/1), and thus (Z)-1-[1-ethyl-7,8-dihydrobenzofuro[4,5-d]thiazol-2(1H)-ylidene]propan-2-one (compound 1) (57.9 mg, 0.222 mmol, 51.3%) was obtained as a light yellow solid. This solid was recrystallized with acetonitrile, so that a light yellow crystal was produced.

TLC R_{f}=0.25 (n-hexane/EtOAc=1/1)

mp 226-227° C.

¹H NMR (500 MHz, CDCl₃) δ 7.29 (d, J=8.5 Hz, 1H), 6.67 (d, J=8.5 Hz, 1H), 5.84 (s, 1H), 4.65 (t, J=8.5 Hz, 2H), 4.12 (q, J=7.0 Hz, 2H), 3.55 (t, J=8.5 Hz, 2H), 2.23 (s, 3H), 1.40 (t, J=7.0 Hz, 3H)

### Production Example 2

### Production of Compound 2

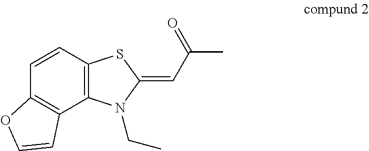 [SMILES: CCN1/C(=C/C(C)=O)Sc2ccc3occc3c21]

A compound 2 was produced in the following manner.

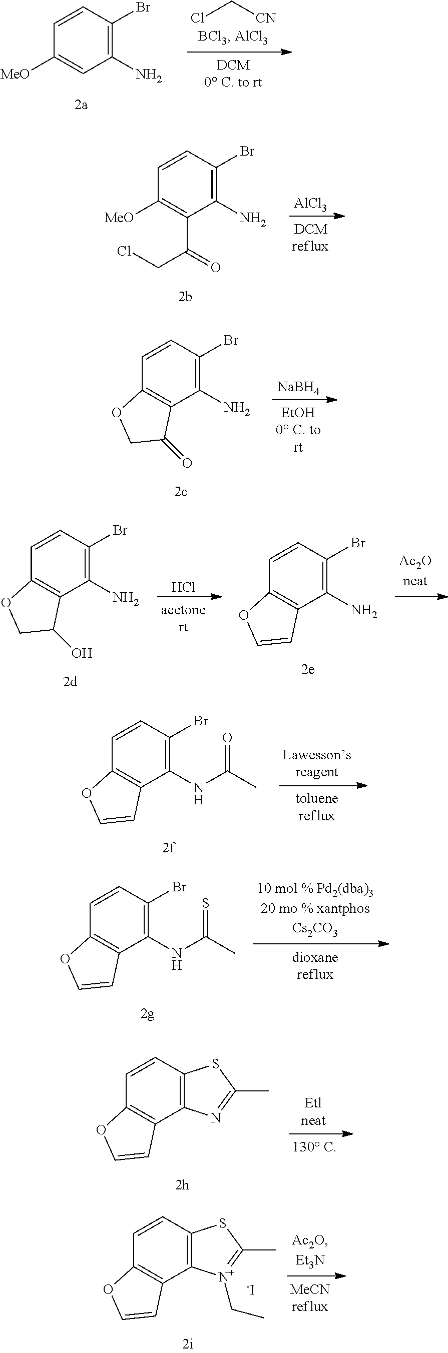

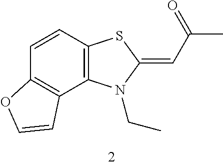 [SMILES: CCN1/C(=C/C(C)=O)Sc2ccc3occc3c21]

### Synthesis of 1-(2-amino-3-bromo-6-methoxyphenyl)-2-chloroethanone (compound 2b)

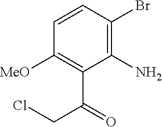 [SMILES: COc1ccc(Br)c(N)c1C(=O)CCl]

A dichloromethane (500 mL) solution of 2-bromo-5-methoxyphenylamine (compound 2a) (100 g, 0.495 mol) was slowly dropped at 0° C. into a dichloromethane (540 mL) solution of boron trichloride (BCl₃) (1M hexane solution, 540 mL, 0.540 mol). The resultant black reaction solution was stirred at 0° C. for 30 minutes, and chloroacetonitrile (76 mL, 1.2 mol) and aluminum chloride (AlCl₃) (72 g, 0.54 mol) were added to the solution. The mixture was stirred at room temperature for 1 hour, and then heated to reflux overnight. After the reaction was completed, the mixture was ice-cooled to 0° C., and hydrochloric acid (2 M, 100 mL) was added to the mixture. Then, hydrochloric acid (5 M, 200 mL) was further added to the mixture and stirred at room temperature for 1 hour. The organic layer was collected and the aqueous layer was extracted with dichloromethane. The combined organic layer was washed with water, dried over sodium sulfate, and filtered. The filtrate was concentrated under reduced pressure, and thus 1-(2-amino-3-bromo-6-methoxyphenyl)-2-chloroethanone (compound 2b) (138 g, 0.495 mol, 100%) was obtained as a dark green solid.

¹H NMR (400 MHz, CDCl₃) δ 7.46 (d, J=8.8 Hz, 1H), 6.74 (brs, 2H), 6.11 (d, J=8.8 Hz, 1H), 4.75 (s, 2H), 3.88 (s, 3H)

### Synthesis of 4-amino-5-bromo-benzofuran-3-one (compound 2c)

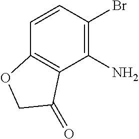 [SMILES: Nc1c(Br)ccc2c1C(=O)CO2]

A dichloromethane (300 mL) solution of the compound 2b (70 g, 0.25 mol) was slowly dropped into a dichloromethane (400 mL, dehydrated) suspension of aluminum chloride (AlCl₃) (100 g, 0.75 mol). The mixture was heated to reflux for 12 hours. After the reaction was completed, the mixture was ice-cooled to 0° C., and hydrochloric acid (2 M) was slowly dropped into the mixture, followed by the addition of methanol and dichloromethane. The organic layer was collected and the aqueous layer was extracted with dichloromethane. The combined organic layer was dried over sodium sulfate and filtered. The filtrate was concentrated under reduced pressure and purified by a silica gel column chromatography, and thus 4-amino-5-bromo-benzofuran-3-one (compound 2c) (30 g, 0.13 mol, 53%) was obtained as a green-brown solid.

¹H NMR (400 MHz, CDCl₃) δ 7.49 (d, J=8.8 Hz, 1H), 6.28 (d, J=8.8 Hz, 1H), 5.78 (brs, 2H), 4.63 (s, 2H)

### Synthesis of 4-amino-5-bromo-2,3-dihydrobenzofuran-3-ol (compound 2d)

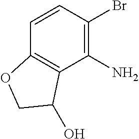 [SMILES: Nc1c(Br)ccc2c1C(O)CO2]

Sodium borohydride (NaBH₄) (47 g, 1.2 mol) was added at 0° C. to an ethanol (EtOH) (3 L) solution of the compound 2c (140 g, 0.614 mol). The temperature was raised to room temperature, and then the mixture was stirred overnight. After the reaction was completed, acetone was added to the mixture and stirred at room temperature for 30 minutes. The mixture was concentrated under reduced pressure. Subsequently, water was added to the mixture, and the mixture was extracted with dichloromethane (1000 mL×2). The combined organic layer was dried over sodium sulfate and filtered. The filtrate was concentrated under reduced pressure, and thus 4-amino-5-bromo-2,3-dihydrobenzofuran-3-ol (compound 2d) was obtained as a colorless solid. This compound was used for the next reaction without purification.

¹H NMR (400 MHz, CDCl₃) δ 7.28 (d, J=8.4 Hz, 1H), 6.18 (d, J=8.4 Hz, 1H), 5.42 (brs, 1H), 4.64-4.60 (m, 1H), 4.42-4.39 (m, 3H), 1.81 (brs, 1H)

### Synthesis of 4-amino-5-bromobenzofuran (compound 2e)

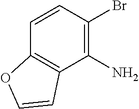 [SMILES: Nc1c(Br)ccc2occc12]

Hydrochloric acid (1M, 100 mL) was added to an acetone solution of the compound 2d (<0.614 mol) and stirred at room temperature for 30 minutes. The mixture was concentrated under reduced pressure, and then diluted with dichloromethane and water. The organic layer was dried over sodium sulfate and filtered. The filtrate was concentrated under reduced pressure, and thus 4-amino-5-bromobenzofuran (compound 2e) was obtained as a yellow solid. This compound was used for the next reaction without purification.

¹H NMR (400 MHz, CDCl₃) δ 7.52 (d, J=2.4 Hz, 1H), 7.30 (d, J=8.8 Hz, 1H), 6.84 (d, J=8.8 Hz, 1H), 6.67 (d, J=2.4 Hz, 1H), 4.33-4.29 (brs, 2H)

### Synthesis of 4-acetamino-5-bromobenzofuran (compound 2)

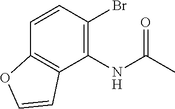

An acetic anhydride (1.5 L) solution of the compound 2e (<0.614 mol) was stirred at room temperature for 2 hours. The precipitated colorless solid was filtered off, and the filtrate was concentrated under reduced pressure. Then, the residue was purified by recrystallization. The solid obtained by the filtration and the solid obtained by the recrystallization were combined and dried, and thus 4-acetamino-5-bromobenzofuran (compound 2) (120 g, 0.47 mol, 77%, for 3 steps) was obtained.

¹H NMR (400 MHz, CDCl₃) δ 7.56 (d, J=2.0 Hz, 1H), 7.49 (brs, 1H), 7.42 (d, J=8.8 Hz, 1H), 7.23 (d, J=8.8 Hz, 1H), 6.73 (d, J=2.0 Hz, 1H), 2.27 (s, 3H)

### Synthesis of 4-(thioacetyl)amino-5-bromobenzofuran (compound 2g)

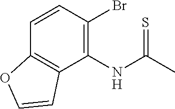

A toluene (2 L) solution of the compound 2f (120 g, 0.472 mol) and Lawesson's reagent (76 g, 0.19 mol) was heated to reflux for 16 hours. After the mixture was allowed to cool to room temperature, the mixture was concentrated under reduced pressure. The residue was purified by a silica gel column chromatography, and thus 4-(thioacetyl)amino-5-bromobenzofuran (compound 2g) (98 g, 0.36 mol, 77%) was obtained as a light yellow solid.

¹H NMR (300 MHz, DMSO-d6) δ 11.60 (brs, 1H), 8.01 (d, J=2.1 Hz, 1H), 7.56 (s, 2H), 6.77 (d, J=2.1 Hz, 1H), 2.66 (s, 3H)

### Synthesis of 2-methyl-7,8-benzofuro[4,5-d]thiazole (compound 2h)

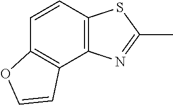

Under the nitrogen atmosphere, the compound 2g (98 g, 0.36 mol) was added to a dioxane (1.5 L) suspension of tris(dibenzylideneacetone)dipalladium (Pd₂(dba)₃) (33 g, 36 mmol), XantPhos (9,9-dimethyl-4,5-bis(diphenylphosphino)xanthene) (41 g, 71 mmol), and cesium carbonate (234 g, 0.72 mol). The mixture was heated to reflux for 16 hours. After the mixture was allowed to cool to room temperature, the mixture was concentrated under reduced pressure. The residue was partially purified (EtOAc) with florisil. The resultant solution was concentrated under reduced pressure and purified by a silica gel column chromatography, and thus 2-methyl-7,8-benzofuro[4,5-d]thiazole (compound 2h) (60 g, 0.32 mol, 88%) was obtained as a yellow solid.

¹H NMR (300 MHz, CDCl₃) δ 7.73 (d, J=2.1 Hz, 1H), 7.67 (d, J=8.7 Hz, 1H), 7.53 (d, J=8.7 Hz, 1H), 7.28 (d, J=2.1 Hz, 1H), 2.90 (s, 3H)

### Synthesis of 1-ethyl-2-methyl-7,8-benzofuro[4,5-d]thiazol-1-ium iodide (compound 2i)

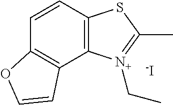

An iodoethane (400 mL) solution of the compound 2h (50 g, 0.26 mol) was tightly sealed and stirred by heating at 130° C. for 50 hours in an autoclave. After the solution was allowed to cool to room temperature, the solution was concentrated under reduced pressure to remove the iodoethane. The residue was suspended in ethyl acetate. This suspension was filtered and the residue was washed with ethyl acetate, and thus 1-ethyl-2-methyl-7,8-benzofuro[4,5-d]thiazol-1-ium iodide (compound 2i) (66 g, 0.19 mol, 74%) was obtained as a green solid.

¹H NMR (400 MHz, DMSO-d6) δ 8.49 (d, J=2.1 Hz, 1H), 8.36 (d, J=8.8 Hz, 1H), 8.16 (d, J=8.8 Hz, 1H), 7.77 (d, J=2.1 Hz, 1H), 4.90 (q, J=7.2 Hz, 2H), 3.26 (s, 3H), 1.53 (t, J=7.2 Hz, 3H)

### Synthesis of (Z)-1-[1-ethyl-7,8-benzofuro[4,5-d]thiazol-2(1H)-ylidene]propan-2-one (compound 2)

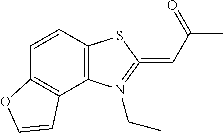

Acetic anhydride (43 mL, 0.46 mol) and triethylamine (80 mL, 0.57 mol) were added to an acetonitrile (250 mL) suspension of the compound 2i (66 g, 0.19 mol). The mixture was heated to reflux for 3 hours. After the mixture was allowed to cool to room temperature, the mixture was concentrated under reduced pressure. The residue was purified by a silica gel column chromatography (petroleum ether/EtOAc=1/1), and thus (Z)-1-[l-ethyl-7,8-benzofuro[4,5-d]thiazol-2(1H)-ylidene]propan-2-one (compound 2) (42 g, 0.16 mol, 84%) was obtained as a yellow solid.

¹H NMR (400 MHz, CDCl₃) δ 7.71 (t, J=1.2 Hz, 1H), 7.44 (d, J=8.8 Hz, 1H), 7.33 (dd, J=8.8, 0.9 Hz, 1H), 6.94 (dd, J=2.1, 0.9 Hz, 1H), 5.92 (s, 1H), 4.27 (q, J=7.2 Hz, 2H), 2.24 (s, 3H), 1.47 (t, J=7.2 Hz, 3H)

### Production Example 3

### Production of Compound 3

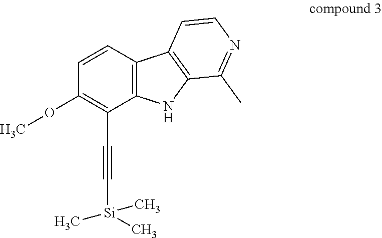

A compound 3 was produced in the following manner.

### Synthesis of compound 3

Under the argon atmosphere, trimethylsilylacetylene (55 L, 0.40 mmol, commercial product) was added at room temperature to a toluene (dehydrated, 2.0 mL)-triethylamine (Et₃N) (2.0 mL) solution including 8-iodoharmine (67.6 mg, 0.200 mmol, synthetic product (US 2007/027199A1)), dichlorobis(triphenylphosphine)palladium ((PPh₃)₂PdCl₂) (7.0 mg, 10 mol, commercial product), copper iodide (CuI) (3.8 mg, 20 μmol, commercial product), and triphenylphosphine (PPh₃) (5.2 mg, 20 μmol, commercial product). The mixture was stirred by heating at 60° C. for 4 hours. After the mixture was allowed to cool to room temperature, a saturated ammonium chloride aqueous solution was added to the mixture. Subsequently, the mixture was extracted with ethyl acetate (×3). The organic layer was dried over anhydrous sodium sulfate, filtered, and then concentrated under reduced pressure. The residue was purified by a silica gel column chromatography (ethyl acetate), and thus 8-[2-(trimethylsilyl)ethynyl]harmine (compound 3) (35.8 mg, 0.116 mmol, 58.0%) was obtained as a colorless solid.

TLC R_{f}=0.40 (ethyl acetate)

mp 185-186° C.

¹H NMR (CDCl₃, 500 MHz) δ 0.37 (s, 9H, Si(CH₃)₃), 2.83 (s, 3H, CH₃), 4.02 (s, 3H, OCH₃), 6.87 (d, J=8.5 Hz, 1H, aromatic), 7.70 (d, J=5.0 Hz, 1H, aromatic), 7.98 (d, J=8.5 Hz, 1H, aromatic), 8.12 (brs, 1H, NH), 8.35 (brs, 1H, aromatic) ¹³C NMR (CDCl₃, 126 MHz) δ 0.3 (3C), 20.2, 56.6, 94.8, 96.9, 104.3, 104.7, 112.4, 115.8, 123.2, 128.9, 134.4, 139.5, 141.3, 142.9, 161.0IR (cm⁻¹) 775, 945, 1099, 1339, 1450, 1614, 2146, 2767, 2861, 2977

### Experimental Example 1

### Neurogenesis in Dentate Gyrus of Hippocampus of Mice

The effect of the compound 2 on neurogenesis in animal individuals (rodents) was studied. The subgranular zone of the dentate gyrus of the hippocampus is a region where neurogenesis occurs. In this region, 5-bromo-2′-deoxyuridine (BrdU) was used as a cell proliferation marker to specifically detect proliferating cells and quantitatively compare the number of proliferating cells. Specifically, the experiments were performed in the following manner.

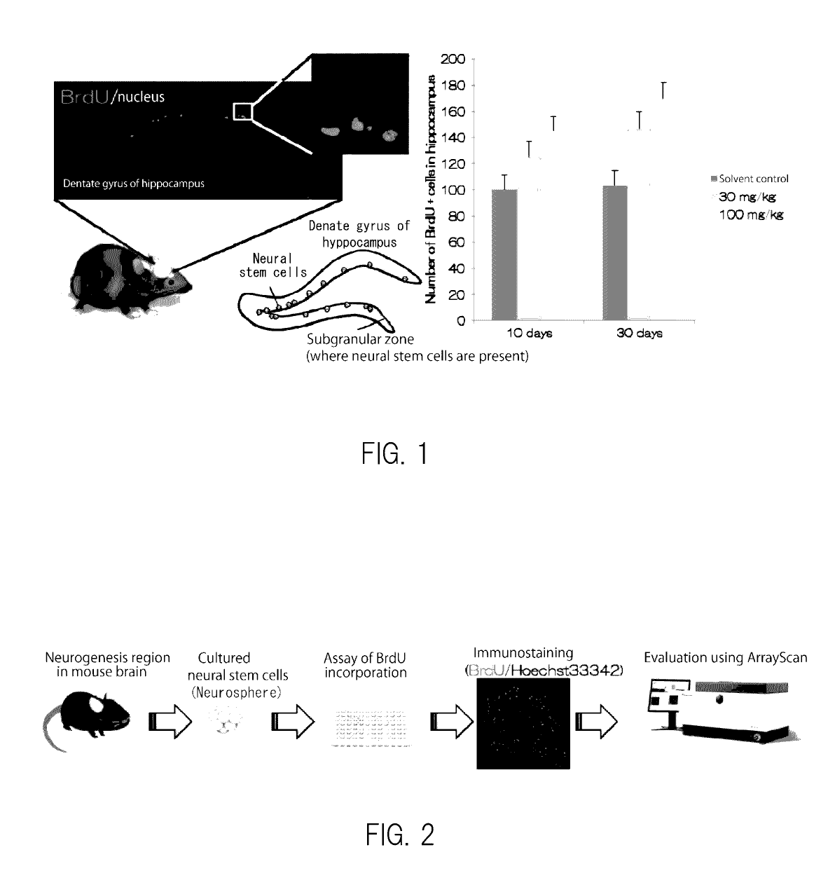

The compound 2 was prepared with a carboxymethyl cellulose solvent and orally administered repeatedly to 9-week-old male C57BL/6J mice (each weighing 25 g) in a dosage of 30 mg/kg and 100 mg/kg for 10 or 30 days. On the last day of the administration, 150 mg/kg of BrdU were intraperitoneally injected into the mice, and the samples were collected 24 hours later by perfusion fixation. Then, coronal sections of 50 μm were prepared by a microtome. After the denaturation of DNA by 1.5 N hydrochloric acid, an anti-BrdU antibody was used to detect BrdU that had been incorporated into the proliferating neural stem cells. Each section was observed with a microscope, and the number of BrdU positive cells per hippocampus was quantified and compared between the administration groups (see FIG. 1).

As shown in FIG. 1, it was confirmed that the number of proliferating cells in the subgranular zone of the dentate gyrus of the hippocampus depended on the dosage and was significantly large in the group of the mice to which the compound 2 was administered, compared to the group of the mice to which the solvent control was administered. Thus, the compound 2 acted on the neural stem cells in the dentate gyrus of the hippocampus of the animal individuals (rodents) and significantly promoted neurogenesis.

### Experimental Example 2

### Effect on Cultured Neural Stem Cells

The study was conducted to investigate whether the compounds 1 to 3 promoted even the growth of cultured neural stem cells that had been isolated from living organisms. Specifically, the experiments were performed in the following manner.

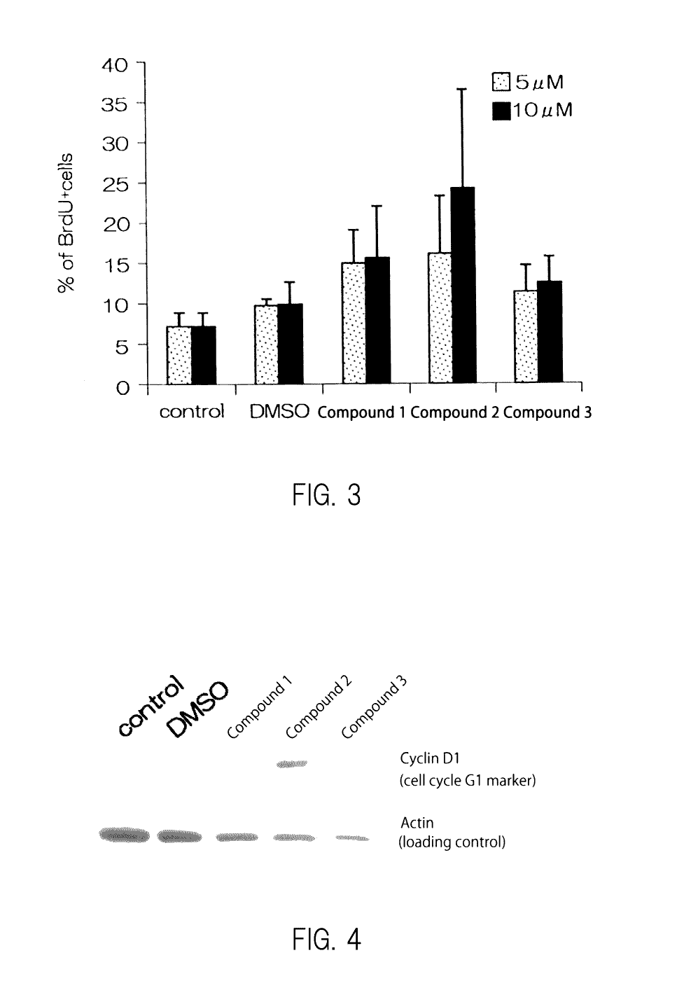

Cells separated from the fetal mouse brain were cultured in suspension in a serum-free culture medium including growth factors such as EGF and bFGF, and then a mass of neural stem cells was isolated. The isolated neural stem cells were cultured in the presence of the compounds 1 to 3 and BrdU (cell proliferation marker), and the proliferating cells were labeled. After the cells were fixed, BrdU was detected by an anti-BrdU antibody, and the ratio of the proliferating cells that had incorporated the BrdU was quantitatively analyzed by ArrayScan (manufactured by Thermo Fisher Scientific Inc.) (see FIGS. 2 and 3). Moreover, the neural stem cells were cultured in the presence of the compounds 1 to 3, and the expression level of cyclin D1, which is a protein for regulating the cell proliferation positively, was detected by western blotting (see FIG. 4).

As shown in FIG. 3, the treatment of the cells with the compounds 1 to 3 increased the amount of incorporation of BrdU (cell proliferation marker). As shown in FIG. 4, the treatment of the cells with the compounds 1 to 3 increased the expression level of the cyclin D1, which is a protein for regulating the cell proliferation positively. Consequently, the compounds 1 to 3 activated the growth of cultured neural stem cells.

### Experimental Example 3

### Inhibitory Effect on DYRK Family and CLK Family

The study was conducted to investigate whether the compounds 1 to 3 inhibited the DYRK activity and the CLK activity in vitro and in vivo. Specifically, the experiments were performed in the following manner.

An in vitro assay of the inhibition of protein phosphorylation activity was performed to investigate whether the compound 1 had a capacity to inhibit the activities of the DYRK family and the CLK family. The results of the assay showed that the compound 1 inhibited 98% of the DYRK1A activity, 99% of the DYRKIB activity, 100% of each of the DYRK2 activity and the DYRK3 activity, 99% of the CLK1 activity, 98% of the CLK2 activity, and 80% of the CLK3 activity.

Moreover, the study was conducted to investigate whether the compound 3 inhibited the DYRK activity in the cells in vivo by using phosphorylation of tau protein (substrate) as a marker. First, cell lines that can induce the expression of DYRK1A and a tau protein by a drug were established. The cell lines were subjected to the drug treatment to induce the expression of DYRKIA and the tau protein. Then, the cells were treated with the compound 3 (10 M) for 4 hours. After the cells were collected, the phosphorylation of the tau protein was detected by western blotting. The results confirmed that the treatment of the cells with the compound 3 inhibited the tau phosphorylation.

Further, the study was conducted to investigate whether the compounds 1 to 2 inhibited the DYRK activity in the animal individuals in vivo by using phosphorylation of tau protein due to acute cold water stress as a marker. The animals were exposed to cold water stress for 10 minutes, and their brains were collected 5 minutes later. Then, the phosphorylation of the tau protein in the brains was confirmed by western blotting. Consequently, the phosphorylation of the tau protein was enhanced by the acute cold water stress. When the compound 1 or the compound 2 was administered to the animal individuals 30 minutes before they underwent the cold water stress, the tau phosphorylation caused by the cold water stress was inhibited.

The in vitro assay of the inhibition of protein phosphorylation activity showed that the compound 1 had the capacity to inhibit both the DYRK activity and the CLK activity. The assay of the phosphorylation of tau in the cells showed that the compound 3 inhibited the tau phosphorylation caused by DYRK. Moreover, the in vivo experiment of the acute cold water stress model in the animal individuals showed that the compound 1 and the compound 2 inhibited the tau phosphorylation. The above results demonstrated that the compounds 1 to 3 had the capacity to inhibit the phosphorylation activity of the DYRK family or the CLK family in vitro/in vivo.

### Experimental Example 4

### Effect of Suppressing DYRKIA Expression on Neurogenesis

The effect of specifically suppressing the expression of DYRK1A that belongs to the DYRK family on neurogenesis was studied by using cultured neural stem cells. Specifically, the experiments were performed in the following manner.

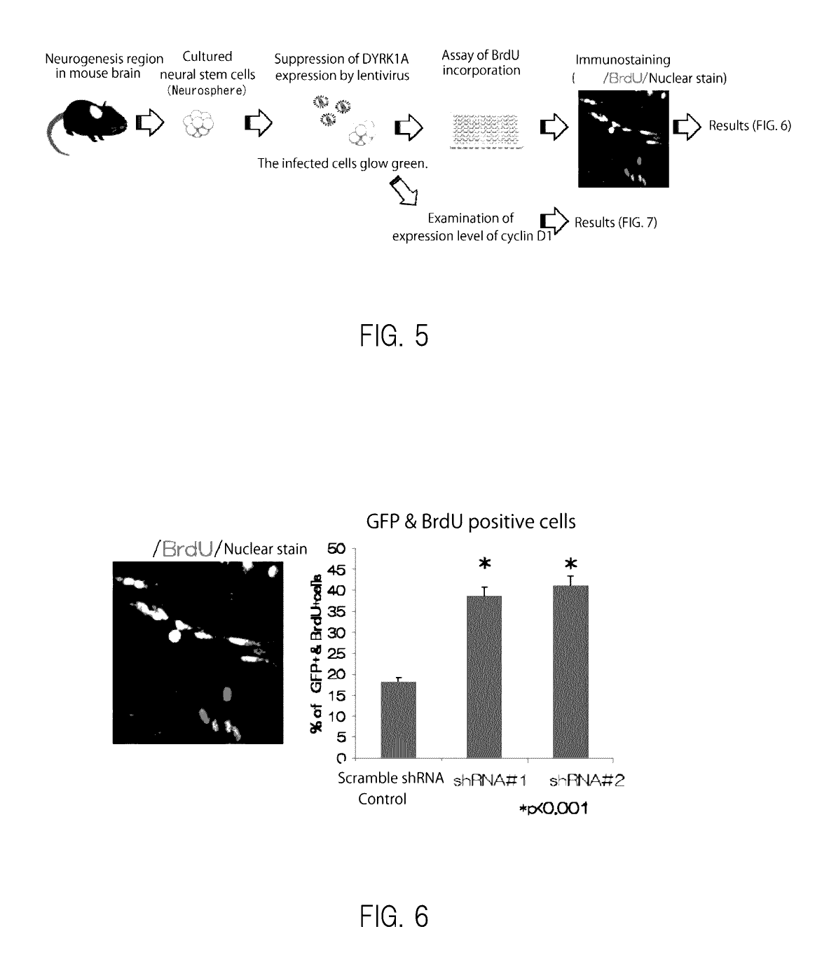

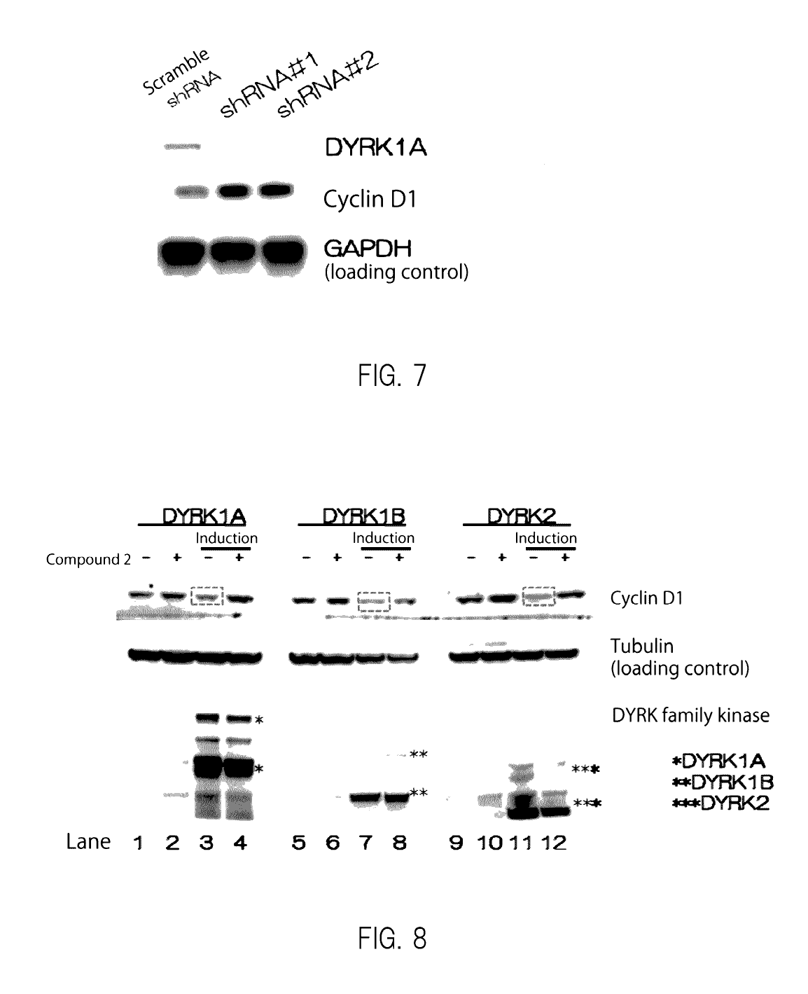

Cells separated from the fetal mouse brain were cultured in suspension in a serum-free culture medium including growth factors such as EGF and bFGF, and then a mass of neural stem cells was isolated. The isolated and cultured neural stem cells were infected with lentivirus expressing short-hairpin RNA (shRNA) that induces the decomposition of mRNA of DYRK1A (see FIG. 5). The cultured neural stem cells, in which the expression of DYRK1A was suppressed, were cultured in the presence of BrdU (cell proliferation marker), and the proliferating cells were labeled. After the cells were fixed, the ratio of the proliferating cells that had incorporated the BrdU was quantitatively analyzed by staining with an anti-BrdU antibody (see FIGS. 5 and 6). Moreover, in the cultured neural stem cells, in which the expression of DYRK1A was suppressed, the expression level of cyclin D1, which is a protein for regulating the cell proliferation positively, was detected by western blotting (see FIGS. 5 and 7).

As shown in FIG. 6, the amount of incorporation of BrdU (cell proliferation marker) was increased in the cultured neural stem cells, in which the expression of DYRK1A was suppressed. As shown in FIG. 7, the expression level of cyclin D1, which is a positive growth factor, was increased in the cultured neural stem cells, in which the expression of DYRK1A was suppressed. The above results demonstrated that the growth of the cultured neural stem cells was activated by suppressing the expression of DYRKIA. In other words, neurogenesis can be activated by inhibiting the DYRK activity.

### Experimental Example 5

### Effect of Inducing DYRK Expression and Inhibiting DYRK Activity on Cell Proliferation

Using cell lines that can induce the expression of DYRKIA, DYRKIB, and DYRK2 by the addition of a drug, the expression level of cyclin D1 was examined when DYRK was overexpressed (see FIG. 8). Specifically, the experiments were performed in the following manner.

A HEK293 Flp-In cell system was used to prepare cell lines that can induce the expression of each of DYRKIA, DYRK1B, and DYRK2 of the DYRK family by the addition of a drug. The cells were treated with the drug for 16 hours to induce the expression of DYRK, and then cultured in the presence of the compound 2 (5 μM) for 4 hours. After the cells were collected, the expression level of cyclin D1 was analyzed by western blotting (see FIG. 8).

As indicated by the lanes 2, 6, and 10 in FIG. 8, the treatment of the cells with the compound 2 inhibited the DYRK activity, and thus increased the expression level of cyclin D1, which is a positive growth factor. As indicated by the lanes 3, 7, and 11 (surrounded by a broken line) in FIG. 8, the expression level of cyclin D1 was reduced by inducing the expression of DYRK. As indicated by the lanes 4, 8, and 12 in FIG. 8, a reduction in cyclin D1 by inducing the expression of DYRK was corrected, since the treatment of the cells with the compound 2 inhibited the DYRK activity. The above results demonstrated that the expression level of cyclin D1 was reduced by inducing the expression of DYRK, and that the expression level of cyclin D1 was increased by inhibiting the DYRK activity. In other words, DYRK is allowed to control the expression level of cyclin D1, thereby activating the cell proliferation.

1. A composition for activating neurogenesis or growth of neurons, comprising as an active ingredient a compound with a DYRK inhibitory capacity or a prodrug thereof or a pharmaceutically acceptable salt thereof.

2. The composition according to claim 1, wherein the compound or the prodrug thereof or the pharmaceutically acceptable salt thereof as the active ingredient further has a CLK inhibitory capacity.

3. A composition for activating neurogenesis or growth of neurons, comprising as an active ingredient a compound expressed by the following general formula (I) and/or a prodrug thereof or a pharmaceutically acceptable salt thereof: 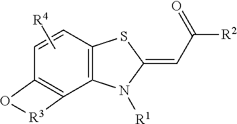
(where, in the general formula (I), R¹ and R² each independently represent a hydrogen atom or a C₁₋₆ hydrocarbon chain,
R³ represents 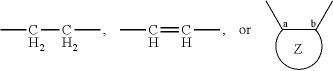
where Z and atoms marked with a and b form a ring selected from the group consisting of one benzene ring, one heteroaromatic ring, an aromatic ring in which one or more benzene rings are condensed, a heteroaromatic ring in which one or more heteroaromatic rings are condensed, a mixed condensed polycyclic ring in which one or more benzene rings are condensed with one or more heteroaromatic rings, and a cyclic aliphatic, and the ring may have at least one substituent that is a hydrogen atom, a halogen atom, or a C₁₋₆ alkyl group, and
R⁴ represents a hydrogen atom, a halogen atom, or a C₁₋₆ alkyl group.

4. The composition for activating neurogenesis or growth of neurons according to claim 3, comprising as an active ingredient a compound expressed by 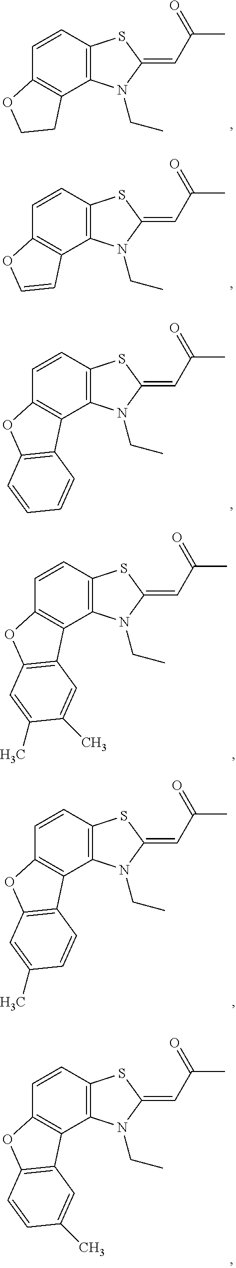 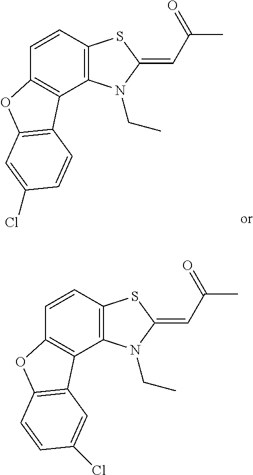 [SMILES: CCN1/C(=C/C(C)=O)Sc2ccc3oc4cc(Cl)ccc4c3c21 ; CCN1/C(=C/C(C)=O)Sc2ccc3oc4ccc(Cl)cc4c3c21]
or a prodrug thereof or a pharmaceutically acceptable salt thereof.

5. The composition for activating neurogenesis or growth of neurons according to claim 14, comprising as an active ingredient a compound expressed by 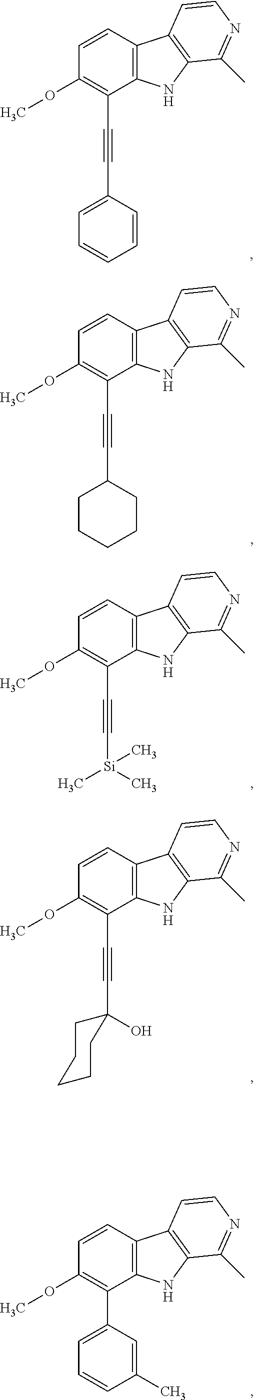 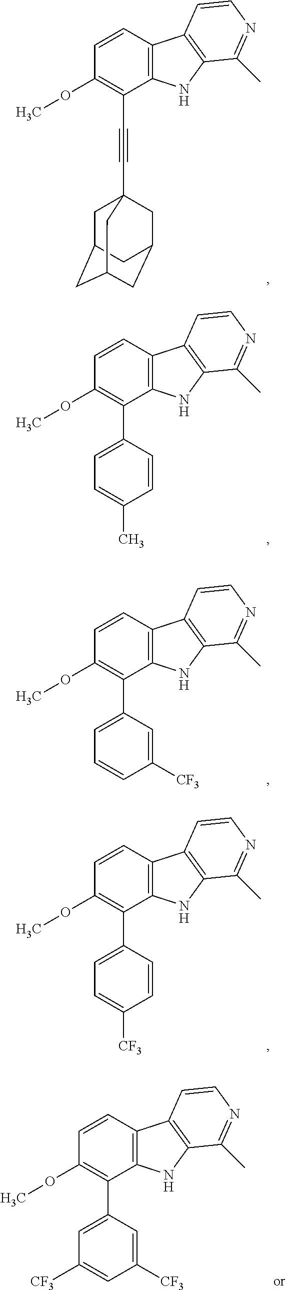 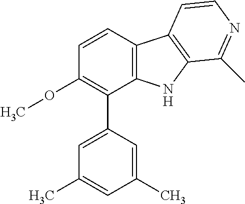
or a prodrug thereof or a pharmaceutically acceptable salt thereof.

6. The composition according to claim 3, wherein the active ingredient has a DYRK inhibitory capacity.

7. The composition according to claim 3, wherein the active ingredient further has a CLK inhibitory capacity.

8. The composition according to claim 3, wherein the composition is a pharmaceutical composition.

9. (canceled)

10. (canceled)

11. A method for activating neurogenesis, comprising:
administering the composition according to claim 3 to a subject.

12. A method for activating growth of neurons, comprising:
culturing neurons in a culture medium containing the composition according to claim 3.

13. A method for preparing neurons or neural stem cells, comprising:
culturing neurons in a culture medium containing the composition according to claim 3.

14. A composition for activating neurogenesis or growth of neurons, comprising as an active ingredient a compound expressed by the following general formula (II) or a prodrug thereof or a pharmaceutically acceptable salt thereof: 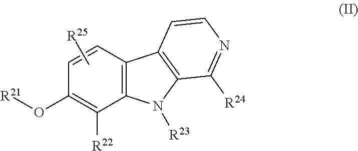
(where, in the general formula (II), R²¹ and R²³ each independently represent a hydrogen atom, a linear, branched, or cyclic C₁₋₆ alkyl group, a benzyl or heteroarylmethyl group, a substituted or unsubstituted aryl group, or a substituted or unsubstituted heteroaryl group,
R²² is selected from the group consisting of —R²⁶, —C≡C—R²⁶, —CH═CH—R²⁶, and —O—(CH₂)n-R²⁶, where n is 1 to 6, R²⁶ is selected from the group consisting of a hydrogen atom, a hydroxyl group, a C₁₋₈ alkyl group, —Si(R²⁷)₃, a substituted or unsubstituted phenyl group, a monocyclic heteroaromatic ring group, and a cyclic aliphatic group, R²⁷ represents a hydrogen atom, a C₁₋₆ alkyl group, a trihalomethyl group, or a hydroxyl group, and three elements represented by R²⁷ of —Si(R²⁷)₃ may differ from each other, alternatively R²² is bonded with R²¹ to form a ring, and —R²¹-R²²— is selected from the group consisting of —(CH₂)m-CH₂—, —CH═CH—, —(CH₂)m-O—, halogen-substituted —(CH₂)m-CH₂—, halogen-substituted —CH═CH—, and halogen-substituted —(CH₂)m-O—, where m is 1 to 6, and
R²⁴ and R²⁵ represent a hydrogen atom or a C₁₋₆ alkyl group).

15. The composition for activating neurogenesis or growth of neurons according to claim 14, wherein R²⁶ is selected from the group consisting of a hydroxyl group, —Si(R²⁷)₃, a substituted or unsubstituted phenyl group, a monocyclic heteroaromatic ring group, and a cyclic aliphatic group.

16. The composition for activating neurogenesis or growth of neurons according to claim 14, wherein R²⁶ is selected from the group consisting of —Si(R²⁷)₃, a substituted or unsubstituted phenyl group, a monocyclic heteroaromatic ring group, and a cyclic aliphatic group.

17. The composition according to claim 14, wherein the active ingredient has a DYRK inhibitory capacity.

18. The composition according to claim 14, wherein the active ingredient further has a CLK inhibitory capacity.

19. The composition according to claim 14, wherein the composition is a pharmaceutical composition.

20. A method for activating neurogenesis, comprising:
administering the composition according to claim 14, to a subject.

21. A method for activating growth of neurons, comprising:
culturing neurons in a culture medium containing the composition according to claim 14.

22. A method for preparing neurons or neural stem cells, comprising:
culturing neurons in a culture medium containing the composition according to claim 14.
